(19) United States
(12) Patent Application Publication
(10) Pub. No.: US 2026/0290498 A1
(43) Pub. Date: Sep. 24, 2026

(54) UTILIZING MOLECULAR DYNAMICS SIMULATION MODELS TO DETERMINE BINDING FREE ENERGY METRICS FROM A CORRECTED PROTEIN BINDING STRUCTURE CONFORMATION AND A CORRECTED COMPOUND BINDING STRUCTURE

(71) Applicant: Recursion Pharmaceuticals, Inc., Salt Lake City, UT (US)

(72) Inventors: Gail Joy BARTLETT, Bristol (GB); Geoffrey Paul Farra WOOD, Mystic, CT (US); Prudencio Murphy Akouété TOSSOU, Montreal (CA); Richard Thomas BRADSHAW, London (GB); Stephan THALER, Montreal (CA); William George GLASS, Maidenhead (GB); Zhiyi WU, Durham (GB)

(21) Appl. No.: 19/436,903

(22) Filed: Dec. 30, 2025

Related U.S. Application Data
(60) Provisional application No. 63/773,745, filed on Mar. 18, 2025.
(60) Provisional application No. 63/867,168, filed on Aug. 20, 2025.

(51) Int. Cl. G16B 15/30; G16B 40/00

(52) U.S. Cl. CPC G16B 15/30; G16B 40/00

(57) ABSTRACT

The present disclosure relates to systems, non-transitory computer-readable media, and methods that that generate binding free energy metrics from protein representations and compound representations. For example, the disclosed systems can utilize a protein-ligand structural generation machine learning model to generate a predicted protein binding structure conformation and a predicted compound binding pose. Further, the disclosed systems can utilize a protein preparation model to generate a corrected compound binding structure based on the predicted protein binding structure conformation. In addition, the disclosed systems can utilize a compound modification model to generate a corrected compound binding structure based on the predicted compound binding pose. The disclosed systems can further utilize a molecular dynamics simulation model to determine a binding free energy metric between the corrected compound binding structure and the corrected protein binding structure conformation.

### CROSS REFERENCE TO RELATED APPS

This application claims the benefit of, and priority to, U.S. Provisional Application No. 63/773,745, entitled “Optimizing Absolute Binding Free Energy Calculations for Production Usage,” filed Mar. 18, 2025, the contents of which are incorporated by reference herein in their entirety. Additionally, this application claims the benefit of, and priority to, U.S. Provisional Application No. 63/867,168, entitled “Boltz-ABFE: Free Energy Perturbation Without Crystal Structures,” filed Aug. 20, 2025, the contents of which are incorporated by reference herein in their entirety.

### BACKGROUND

Recent years have seen significant developments in hardware and software platforms for utilizing machine learning tools to simulate binding reactions between a compound and a protein. Although conventional systems can simulate binding reactions between a protein and a compound, they suffer from a number of technical deficiencies with regard to accuracy, computational efficiency, and operational flexibility, especially those pertaining to the technical field of compound exploration and discovery.

### SUMMARY

Embodiments of the present disclosure provide benefits and/or solve one or more of the foregoing problems in the art with systems, non-transitory computer-readable media, and methods for simulating binding reactions between a protein and compound (e.g., utilizing a protein representation and a compound representation) and correcting protein and compound structures to generate a binding free energy metric for the binding simulation. For example, the disclosed systems can utilize a protein-ligand structural generation machine learning model to generate a binding simulation between a protein and a compound by generating a predicted protein binding structure conformation and a predicted compound binding pose for the protein and the compound. Further, the disclosed systems can augment or otherwise improve the predicted protein binding structure conformation and the predicted compound binding pose, such as by utilizing a protein preparation model to generate a corrected protein binding structure conformation from the predicted protein binding structure conformation and/or utilizing a compound modification model to generate a corrected compound binding structure from the predicted compound binding pose. Moreover, the disclosed systems can utilize the corrected protein binding structure conformation and the corrected compound binding structure to simulate, via a molecular dynamics simulation model, a molecular dynamics reaction and determine a binding free energy metric between the protein and the compound according to simulated interactions between the corrected protein binding structure conformation and the corrected compound binding structure.

Additional features and advantages of one or more embodiments of the present disclosure are outlined in the description which follows, and in part will be obvious from the description, or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview of a binding energy modeling system initiating a compound-target discovery pipeline based on generating a binding free energy metric between a corrected protein binding structure conformation and a corrected compound binding structure in accordance with one or more embodiments.

FIG. 2 illustrates a binding energy modeling system utilizing a protein-ligand structural generation machine learning model to generate a binding simulation for a protein and a compound by generating a predicted protein binding structure conformation and a predicted compound binding pose in accordance with one or more embodiments.

FIG. 3 illustrates a binding energy modeling system utilizing a protein preparation model to generate a corrected protein binding structure conformation based on a predicted protein binding structure conformation in accordance with one or more embodiments.

FIG. 4 illustrates a binding energy modeling system utilizing a compound modification model and a re-docking simulation model to generate a corrected compound binding structure based on a predicted compound binding pose in accordance with one or more embodiments.

FIGS. 5A-5B illustrate a binding energy modeling system generating different compound representations having different stereochemistries and comparing the different compound representations to a predicted protein binding structure conformation to determine a corrected compound binding structure based on corresponding stereochemistry error metrics in accordance with one or more embodiments.

FIG. 6 illustrates a binding energy modeling system using a molecular dynamics simulation model to apply a corrective force to an unstable bond angle to generate a binding free energy metric in accordance with one or more embodiments.

FIG. 7 illustrates a binding energy modeling system generating a potency metric for a query compound and performing actions within a compound-target discovery pipeline based on the potency metric in accordance with one or more embodiments.

FIG. 8 illustrates a schematic diagram of a system environment in which a binding energy modeling system can operate in accordance with one or more embodiments.

FIG. 9 illustrates an example series of acts for generating a binding free energy metric from a corrected protein binding structure conformation and a corrected compound binding structure in accordance with one or more embodiments.

FIG. 10 illustrates a block diagram of a computing device for implementing one or more embodiments of the present disclosure.

### DETAILED DESCRIPTION

This disclosure describes one or more embodiments of a binding energy modeling system that can utilize machine learning models to generate binding free energy metrics from protein representations and compound representations. In one or more embodiments, the binding energy modeling system utilizes a protein-ligand structural generation machine learning model to generate a binding simulation between a predicted protein binding structure conformation and a predicted compound binding pose. Further, the binding energy modeling system can augment or otherwise modify the predicted protein binding structure conformation and the predicted compound binding pose. To illustrate, the binding energy modeling system can utilize a protein preparation model to generate a corrected (e.g., chemically valid) protein binding structure conformation based on the predicted protein binding structure conformation. Specifically, the binding energy modeling system can generate the corrected protein binding structure conformation by utilizing the protein preparation model to add hydrogen atom representations and/or electron representations to the predicted protein binding structure conformation.

Further, the binding energy modeling system can use a compound modification model to generate a corrected compound binding structure based at least in part on the predicted compound binding pose. For example, the binding energy modeling system can utilize the compound modification model to identify one or more errors in the predicted compound binding pose, such as a bond order error, an aromaticity error, and/or a stereochemistry error. The binding energy modeling system can generate a modified representation of the predicted compound binding pose to correct the one or more errors. The binding energy modeling system can further utilize a re-docking simulation model to simulate docking the modified representation of the predicted compound binding pose to the predicted protein binding structure conformation and/or to the corrected protein binding structure conformation.

Additionally, the binding energy modeling system can analyze the corrected protein binding structure conformation and the corrected compound binding utilizing a molecular dynamics simulation model to determine a binding free energy metric between the corrected protein binding structure conformation and the corrected compound binding structure. In some embodiments, the binding energy modeling system can identify an unstable bond angle between the corrected protein binding structure conformation and/or the corrected compound binding structure. Specifically, the binding energy modeling system can determine that the unstable bond angle destabilizes operations within the molecular dynamics simulation model. The binding energy modeling system can utilize the molecular dynamics simulation model to apply a corrective force to the unstable bond angle to prevent operational instability within the molecular dynamics simulation model. The binding energy modeling system can generate the binding free energy metric based on applying the corrective force to the unstable bond angle.

Further, in some embodiments the binding energy modeling system initiates or otherwise modifies operations within a compound-target discovery pipeline based on determining the binding free energy metric. For example, the binding energy modeling system can perform additional drug discovery assays based on the binding free energy metric. Similarly, the binding energy modeling system can select a compound for a drug discovery pipeline based on the binding free energy metric. Moreover, the binding energy modeling system can initiate clinical trials and provide therapeutic treatment for a compound selected utilizing the binding free energy metric.

In some implementations, the binding energy modeling system determines a potency metric for a compound (e.g., such as the compound that the binding energy modeling system generates representations of) based on applying the compound to a plurality of cells in a plurality of concentrations (e.g., based on performing a compound perturbation using the compound). In some embodiments, the binding energy modeling system generates embeddings to represent effects of the compound on cells and determines a potency metric from the embeddings. The binding energy modeling system can compare the potency metric to the binding free energy metric to determine to initiate further downstream operations within a tech-bio exploration system.

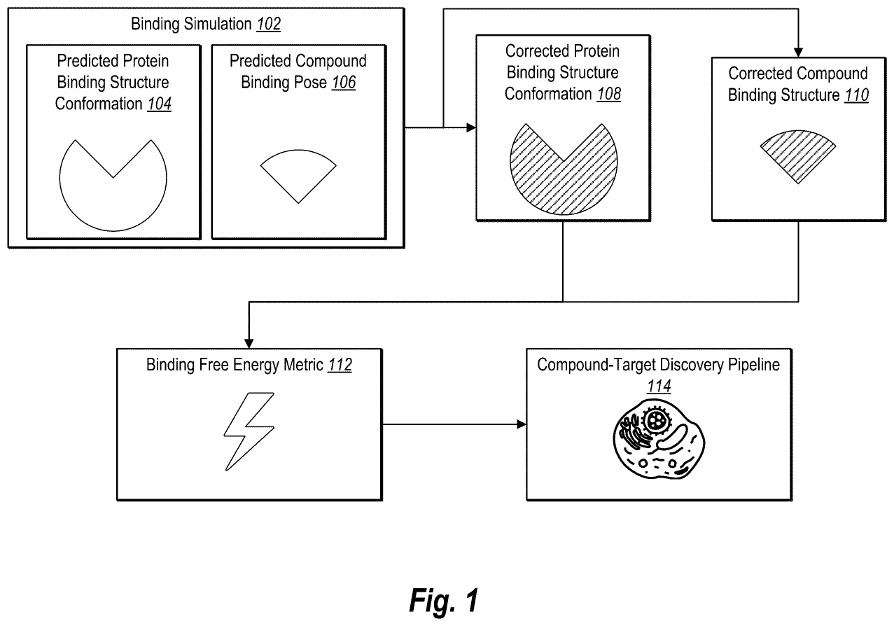

As previously mentioned, the binding energy modeling system 100 can generate a binding free energy metric from a corrected protein binding structure conformation and a corrected compound binding structure. FIG. 1 illustrates the binding energy modeling system 100 initiating a compound-target discovery pipeline base on generating a binding free energy metric in accordance with one or more embodiments.

As shown in FIG. 1, the binding energy modeling system 100 can generate a binding simulation 102 for a compound (e.g., such as a ligand) and a protein. Indeed, the binding energy modeling system 100 can generate the binding simulation 102 to include a predicted protein binding structure conformation 104 for the protein. Moreover, the binding energy modeling system 100 can generate the binding simulation 102 to include a predicted compound binding pose 106 for the compound. For example, the binding energy modeling system 100 can generate the predicted protein binding structure conformation 104 and the predicted compound binding pose 106 to be complementary representations of complementary structures (e.g., for the protein and the compound) that would facilitate or otherwise lead to a binding reaction between the compound and the protein. In some embodiments, the binding energy modeling system 100 utilizes a protein-ligand structural generation machine learning model to generate the binding simulation 102. Further, in some embodiments the binding energy modeling system 100 identifies the protein and/or the compound based on receiving a query from a client device.

As shown in FIG. 1, based on generating the binding simulation 102, the binding energy modeling system 100 can generate a corrected protein binding structure conformation 108. Indeed, the binding energy modeling system 100 can generate the corrected protein binding structure conformation 108 by identifying one or more protein inaccuracies in the predicted protein binding structure conformation 104. In some embodiments, the binding energy modeling system 100 utilizes a protein preparation model to generate the corrected protein binding structure conformation 108.

Further, based on generating the binding simulation 102, the binding energy modeling system 100 can generate a corrected compound binding structure 110. For example, the binding energy modeling system 100 can utilize a compound modification model to identify one or more compound invalidities of the predicted compound binding pose 106 and can generate the corrected compound binding structure 110 to remedy the compound invalidities.

As illustrated in FIG. 1, the binding energy modeling system 100 can determine a binding free energy metric 112 between the corrected protein binding structure conformation 108 and the corrected compound binding structure 110. For example, the binding energy modeling system 100 can utilize a molecular dynamics simulation model to perform a binding simulation (e.g., such as a binding reaction) between the corrected protein binding structure conformation 108 and the corrected compound binding structure 110. The binding energy modeling system 100 can utilize the molecular dynamics simulation model to determine the binding free energy metric 112 by determining a difference in free energy between a bound state and an unbound state (indicating how strongly a ligand and protein bind together).

As shown in FIG. 1, the binding energy modeling system 100 can utilize the binding free energy metric 112 to initiate a compound-target discovery pipeline 114. For example, the binding energy modeling system 100 can initiate the compound-target discovery pipeline 114 by initiating one or more experimental workflows based on the protein and/or the compound (e.g., in vitro experiments such as compound discovery assays, ADMET studies, pharmacokinetics studies, biophysical binding validation, among others). In some embodiments, the binding energy modeling system 100 initiates the compound-target discovery pipeline 114 based on determining the binding free energy metric 112 meets an energy release threshold.

As previously mentioned, conventional systems suffer from a number of technical deficiencies, particularly with regard to accuracy, computational efficiency, and operational flexibility of implementing computing systems, especially those pertaining to the technical fields of drug discovery and artificial intelligence. For example, conventional systems utilize a variety of complex technical processes in the drug discovery pipeline to identify, develop, and test compounds. To illustrate, conventional systems utilize computer-implemented techniques for target identification, target validation, hit identification, molecular dynamics simulations, hit-to-lead optimization, lead optimization, preclinical development, and clinical trials. For example, molecular dynamics simulations can include a variety of computer implemented processes to generate protein representations and compound representations, simulate binding reactions between the protein and compound representations, and perform genomic and proteomic analysis, bioinformatics, and disease pathway analysis. These processes are often inaccurate and inefficient.

For example, with regard to molecular dynamics simulations, conventional systems often experience simulation failures due to bond angle instabilities in representations of compounds and/or proteins. To illustrate, conventional systems often select restraint atom representations in the protein representation and/or the compound representation. Conventional systems restrain the movement and/or rotation of the restraint atom representations to prevent movement of the protein representation and/or the compound representation, such as translation or rotation that does not correspond to the binding simulation. However, as the bond angles of the restraint atom representations approach 0° and/or 180°, conventional systems are prone to experience either operational failure or generate inaccurate calculations for the simulation due to impacts of harmonic instabilities that arise from the bond angles approaching 0° or 180°. Indeed, these inaccurate calculations and/or operational failures are due to technological shortcomings of conventional systems, as the bond angles affect the simulation of the binding reaction.

Further, conventional systems often generate inaccurate representations of binding simulations due to generating inaccurate protein representations and/or inaccurate compound representations. For example, conventional systems often generate protein representations that inaccurately represent the charges and/or electronic forces of the protein representation. Additionally, conventional systems often generate compound representations that inaccurately represent the compound, such as by generating compound representations that contain one or more of a bond order error, an aromaticity error, and/or a stereochemistry error. The inaccurate protein representations and compound representations can in turn lead to inaccurate molecular dynamics simulations.

Further, based at least in part on the inaccuracies of conventional systems, conventional systems often undergo time consuming and computationally inefficient processes to generate molecular dynamics simulations. Indeed, when a conventional system experiences an operational failure and/or generates an inaccurate molecular dynamics simulation, the conventional system must expend additional computational resources (e.g., such as processing power, bandwidth, and/or memory) to repeat the molecular dynamics simulation. Similarly, when a conventional system generates an inaccurate molecular dynamics simulation due to errors in protein representations and/or compound representations, the conventional system must expend additional computational resources to identify the errors in the protein and/or compound representations, generate corrected representations, and re-run molecular dynamics simulations based on identifying the errors. Accordingly, conventional systems waste significant amounts of computational resources (and thus create unnecessary systemic overhead) that could otherwise be saved by a more accurate system.

Indeed, the inefficiency of conventional systems is only exacerbated in performing multiple molecular dynamics simulations over time. For example, conventional systems often perform multiple molecular dynamics simulations. Accordingly, experiences of computational failures for molecular dynamics simulations can affect the computational resources available to perform other molecular dynamics simulations in parallel. Further, because conventional systems often re-use or only slightly modify representations of proteins and/or compounds across multiple simulations, errors in the chemical accuracy of the protein and/or compound representations can propagate across multiple molecular dynamics simulations.

Moreover, conventional systems are often operationally inflexible in the ways that they generate binding free energy metrics. Indeed, many conventional systems rely on experimentally-derived data, such as X-ray crystallography data, cryo-electron microstrip data, or nuclear magnetic resonance data to determine atomic coordinates for 3D representations of binding reactions between compounds and proteins. Further, acquiring the necessary experimentally-derived data requires significant amounts of time, fiscal resources, and computational resources, which compounds the above-mentioned issues with regard to computational inefficiency.

As suggested by the foregoing, the binding energy modeling system provides a variety of technical advantages relative to conventional systems. For example, the binding energy modeling system can improve the accuracy of existing technical discovery pipelines by identifying unstable bond angles during molecular dynamics simulations and applying a corrective force to constituents of the unstable bond angle, thus preventing the unstable bond angle from approaching 0° and/or 180° and thus destabilizing the molecular dynamics simulation. Indeed, because the unstable bond angles are a problem that specifically arise in molecular dynamics simulations, by applying the corrective force to prevent the bond angle from reaching 0° and/or 180°, the molecular dynamics simulation model can directly provide technical improvements to the technical field of compound discovery.

Further, in one or more implementations, the binding energy modeling system 100 improves accuracy compared to conventional systems by utilizing both protein-ligand structural generation machine learning models (such as Boltz or Boltz-2) together with molecular dynamics models to generate improved absolute binding free energy predictions. Moreover, in some implementations the binding energy modeling system 100 generates improved predictions relative to conventional systems by augmenting or otherwise modifying predicted protein binding structure conformations and/or predicted compound binding poses to generate corrected protein binding structure conformations and/or corrected compound binding structures. For example, the binding energy modeling system 100 can utilize a protein preparation model to generate, based on the predicted protein binding structure conformation, a corrected protein binding structure conformation that is chemically accurate. Moreover, the binding energy modeling system 100 can utilize a compound modification model to identify one or more errors in a predicted compound binding pose, such as a bond order error, an aromaticity error, and/or a stereochemistry error, and further generate an intermediate compound binding structure (and/or a modified compound binding pose) that corrects the bond order error, the aromaticity error, and/or the stereochemistry error. The binding energy modeling system 100 can further use a re-docking simulation model to simulate docking the intermediate compound binding structure (and/or the modified compound binding pose) to a representation of a protein to determine that the intermediate compound binding structure (and/or the modified compound binding pose) is a corrected compound binding structure. In addition, by improving protein and compound structures, the binding energy modeling system 100 can significantly improve binding free energy metrics generated by molecular dynamics models.

Moreover, based at least in part on the improvements in accuracy compared to conventional systems, the binding energy modeling system 100 improves computational efficiency compared to conventional systems. For example, by applying corrective forces to unstable bond angles during molecular dynamics simulations, the binding energy modeling system 100 reduces instances of simulation error due to computational errors arising from unstable bond angles (e.g., angles that destabilize or otherwise disrupt the computations of a molecular dynamics simulation), thus reducing the amount of computational resources necessary to accurately simulate a molecular dynamics simulation. Moreover, by generating corrected protein binding structure conformations and corrected compound binding structures before performing the molecular dynamics simulation, the binding energy modeling system 100 further reduces instances of inaccurate molecular dynamics simulations, thereby reducing the computational resources required to re-run the molecular dynamics simulations.

Further, the binding energy modeling system 100 improves operational flexibility compared to conventional systems. For example, rather than relying on experimentally-derived data to generate binding free energy metrics, the binding energy modeling system 100 utilizes a molecular dynamics machine learning ensemble that includes one or more of a protein-ligand structural generation machine learning model, a protein preparation model, a compound modification model, a re-docking simulation model, and a molecular dynamics simulation model to generate a binding free energy metric for a protein representation and a compound representation. Indeed, the binding energy modeling system 100 utilizes a protein-ligand structural generation machine learning model to generate a predicted protein binding structure conformation and a predicted compound binding pose. The binding energy modeling system 100 can further utilize a protein preparation model to modify the predicted protein binding structure conformation to generate a chemically valid representation of a protein.

Additionally, the binding energy modeling system 100 can utilize a compound modification model to modify the predicted compound binding pose to generate a chemically valid representation of a compound. The binding energy modeling system 100 can further provide the chemically valid representations of the protein and the compound to a molecular dynamics simulation model to generate the binding free energy metric. Thus, by utilizing the molecular dynamics machine learning model, the binding energy modeling system 100 improves operational flexibility and functionality compared to conventional systems by generating binding free energy metrics from a protein input and a compound input for the protein-ligand structural generation machine learning model as opposed to experimentally-derived structural data.

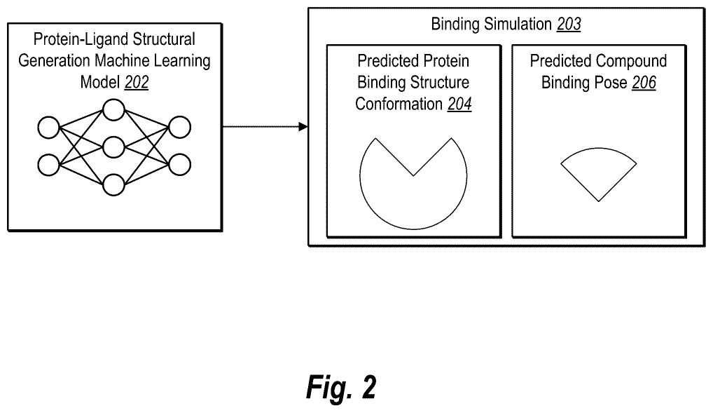

As previously mentioned, the binding energy modeling system 100 can generate a binding simulation for a protein and a compound. FIG. 2 illustrates the binding energy modeling system 100 utilizing a protein-ligand structural generation machine learning model to generate a binding simulation between a predicted protein binding structure conformation and a predicted compound binding pose in accordance with one or more embodiments.

As shown in FIG. 2, the binding energy modeling system 100 can utilize a protein-ligand structural generation machine learning model 202 to generate a binding simulation 203. As used herein, a “machine learning model” can refer to a computer algorithm or a collection of computer algorithms that can be trained and/or tuned based on inputs to approximate unknown functions. For example, a machine learning model can include a computer algorithm with branches, weights, or parameters that changed based on training data to improve for a particular task. Thus, a machine learning model can utilize one or more learning techniques to improve in accuracy and/or effectiveness. Example machine learning models include various types of decision trees (e.g., Lightgbm, XGBoost, and Random Forest), support vector machines, Bayesian networks, random forest models, or neural networks (e.g., deep neural networks).

Along these lines, the term “neural network” refers to a machine learning model that can be trained and/or tuned based on inputs to determine classifications, scores, or approximate unknown functions. For example, a neural network includes a model of interconnected artificial neurons (e.g., organized in layers) that communicate and learn to approximate complex functions and generate outputs based on a plurality of inputs provided to the neural network. In some cases, a neural network refers to an algorithm (or a set of algorithms) that implements deep learning techniques to model high-level abstractions in data. A neural network can include various layers such as an input layer, one or more hidden layers, and an output layer that each perform tasks for processing data. For example, a neural network can include a deep neural network, a convolutional neural network, a recurrent neural network (e.g., an LSTM), a graph neural network, an LLM, or a generative neural network.

Further, as used herein, the term “protein-ligand structural generation machine learning model” can refer to a machine learning model that generates a binding simulation that includes a predicted protein binding structure conformation and a predicted compound binding pose. For example, the binding energy modeling system 100 can provide a protein input and a compound input to the protein-ligand structural generation machine learning model. To illustrate, the protein input can be a static three-dimensional structure that includes atomic coordinates for the protein. Indeed, the protein input can define the base geometry of the protein in an unbound state. For example, the protein input can be a protein data bank file, a macromolecular crystallographic information file, an AlphaFold-predicted structure file, a cryo-EM-derived structure, and/or a homology model, among others.

In some implementations, the protein-ligand structural generation machine learning model 202 can utilize a co-folding model. For example, rather than folding a protein alone and separately docking a ligand, the model folds the interacting complex together (e.g., utilizing a transformer-style backbone). In this manner, the protein-ligand structural generation machine learning model 202 can capture how both partners adapt to each other. The protein-ligand structural generation machine learning model 202 can also utilize an affinity model that analyzes learned embeddings to generate a binding probability. In some embodiments, the protein-ligand structural generation machine learning model 202 can be a geometric deep learning model, such as a Boltz model (e.g., such as Boltz or Boltz 2).

Further, the compound input can be a static representation of a compound. For example, the compound input can be a simplified molecular input line entry system (SMILES) string that is a text-based representation of the compound. To illustrate, the SMILES string can be a linear string of text that represents aspects of the compound, such as constituent atoms, bonds, connectivity, functional groups, and/or stereochemistry, among others. In some embodiments, the compound input is a structure data file, a MOL2 file, a PDBQT file, and/or a pre-generated 3D conformer, among others.

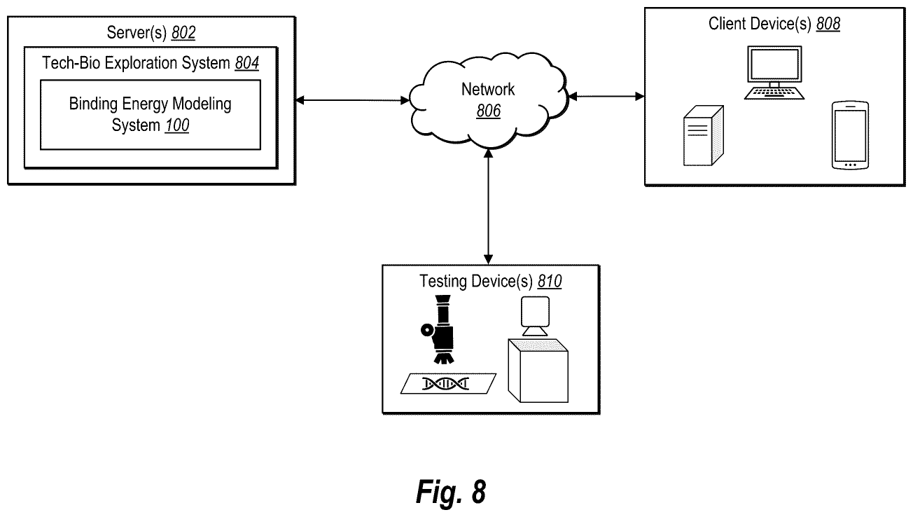

In some embodiments, the binding energy modeling system 100 can determine the protein input for the protein-ligand structural generation machine learning model 202 based on a binding simulation query received from a client device. In some embodiments, the binding energy modeling system 100 autonomously generates the binding simulation query according to an interaction with a tech-bio exploration system (e.g., the tech-bio exploration system 804 of FIG. 8, discussed below). For example, the binding energy modeling system 100 can autonomously generate the binding simulation query based on identifying a phenomic similarity between a compound and a target. For example, the binding energy modeling system 100 can capture a phenomic image of a cell exposed to a perturbation (e.g., a compound or a gene knockout) and utilize a machine learning embedding model to generate a phenomic embedding. The binding energy modeling system 100 can then compare phenomic embeddings of different perturbations to generate similarity measures (e.g., cosine similarity). Based on ranking similarity measures to a particular target, the binding energy modeling system 100 can select a compound and/or protein to analyze utilizing the protein-ligand structural generation machine learning model 202.

As shown, the binding energy modeling system 100 can utilize the protein-ligand structural generation machine learning model 202 to generate a binding simulation 203. As used herein, the term “binding simulation” refers to a representation of a predicted interaction between a compound and a protein. Indeed, the binding energy modeling system 100 can generate the binding simulation 203 to include a predicted protein binding structure conformation 204 and a predicted compound binding pose 206. To illustrate, the binding energy modeling system 100 can generate the binding simulation 203 to depict a prediction of how the compound and protein will orient, conform, and/or interact with each other. In some embodiments, the binding energy modeling system 100 generates the binding simulation 203 to represent a subset of the protein and/or the compound. For example, the binding energy modeling system 100 can generate the binding simulation to include portions of the protein and the compound that interact with each other, but not portions of the protein and/or the compound that are not involved in the binding interaction.

As used herein, the term “predicted protein binding structure conformation” refers to a three-dimensional representation of a compound-dependent conformation of a protein. For example, the binding energy modeling system 100 can generate the predicted protein binding structure conformation to have one or more functional groups whose orientation is dependent upon or otherwise influenced by binding the compound (e.g., surface geometry that is influenced by binding the compound). Indeed, the binding energy modeling system 100 can generate the predicted protein binding structure conformation to represent changes that occur to the protein as a result of binding the compound, such as changes in a backbone of the protein, localized shifts in loop or domain positions, and/or other changes in secondary or tertiary structural elements of the protein to stabilize the binding simulation. In some embodiments, the binding energy modeling system 100 generates the predicted protein binding structure conformation to be a template representation that corresponds to various proteins.

Similarly, as used herein, the term “predicted compound binding pose” refers to a three-dimensional representation of the compound that is dependent on and/or complementary to the predicted protein binding structure conformation. For example, the binding energy modeling system 100 can generate the predicted compound binding pose to indicate a representation of a conformation and/or orientation of the compound in a bound state with the protein. To illustrate, the binding energy modeling system 100 can generate the predicted compound binding pose to represent atomic coordinates of the compound within a binding site of the protein. Further, the binding energy modeling system 100 can generate the predicted compound binding pose to represent conformational strain and/or orientation of functional groups that enables non-covalent interactions with the predicted protein binding structure conformation. Indeed, the binding energy modeling system 100 can generate the predicted compound binding pose to represent structural adaptations of the compound that enable the predicted compound binding pose and the predicted protein binding structure conformation to interact in the binding simulation.

Accordingly, the binding energy modeling system 100 can utilize the protein-ligand structural generation machine learning model 202 to generate the binding simulation 203 as a first pass representation of the binding interaction between the protein and the compound. Indeed, the binding energy modeling system 100 can generate the predicted protein binding structure conformation 204 and the predicted compound binding pose 206 to include atomic coordinates for heavy atoms (e.g., atoms other than hydrogen) of the predicted protein binding structure conformation 204 and the predicted compound binding pose 206.

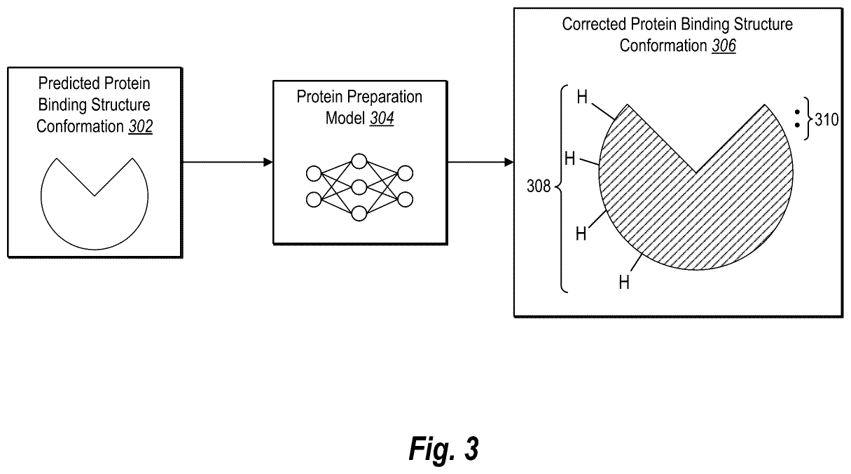

As previously mentioned, the binding energy modeling system 100 can generate a corrected protein binding structure conformation. FIG. 3 illustrates the binding energy modeling system 100 utilizing a protein preparation model to generate a corrected protein binding structure conformation based on a predicted protein binding structure conformation in accordance with one or more embodiments.

As shown in FIG. 3 and as previously discussed, the binding energy modeling system 100 can generate a predicted protein binding structure conformation 302 (e.g., utilizing a protein-ligand structural generation machine learning model). The binding energy modeling system 100 can further provide the predicted protein binding structure conformation 302 to a protein preparation model 304. As used herein, the term “protein preparation model” refers to a machine learning model that the binding energy modeling system 100 trains, finetunes and/or otherwise utilizes to identify and correct errors in a predicted protein binding structure conformation. Indeed, the binding energy modeling system 100 can utilize the protein preparation model to generate chemically valid representations of proteins. To illustrate, the binding energy modeling system 100 can utilize a protein preparation model to add chemical validity features to the predicted protein binding structure conformation. To illustrate, the binding energy modeling system 100 can utilize the protein preparation model 304 to modify the predicted protein binding structure conformation 302 by adding representations of hydrogens and/or electrons, assigning protonation states and/or partial charges, and/or adjusting atomic coordinates of the predicted protein binding structure conformation to reflect a stable and realistic protein conformation that is suitable for use in molecular dynamics simulations.

For example, the protein preparation model can be a protein preparation toolkit (e.g., such as OpenEye, OpenEye Spruce Toolkit, Schrödinger Protein Preparation Wizard, MOE Structure Preparation, ChimeraX DockPrep) that the binding energy modeling system 100 utilizes to preprocess the predicted protein binding structure conformation 302 for molecular dynamics modeling. For example, the binding energy modeling system 100 can utilize the protein preparation model 304 to optimize proton positions of the predicted protein binding structure conformation 302, assign partial charges to the predicted protein binding structure conformation 302, and/or to cap the N-terminus and the C-terminus of the predicted protein binding structure conformation 302, respectively with an acetyl and N-methyl group.

As previously discussed, the binding energy modeling system 100 can utilize the protein preparation model 304 to generate a corrected protein binding structure conformation 306. As used herein, the term “corrected protein binding structure conformation” refers to a chemically valid representation of a protein in a binding conformation that is specific to a compound. For example, the binding energy modeling system 100 can generate the corrected protein binding structure conformation by processing a predicted protein binding structure conformation and adding representations of chemical features to the heavy atom representations.

As shown in FIG. 3, the binding energy modeling system 100 can generate the corrected protein binding structure conformation 306 to include hydrogen atom representations 308. For example, the binding energy modeling system 100 can generate the corrected protein binding structure conformation 306 by adding the hydrogen atom representations 308 to the predicted protein binding structure conformation 302. Additionally, the binding energy modeling system 100 can generate the corrected protein binding structure conformation 306 to include electron representations 310 for the predicted protein binding structure conformation 302.

For example, the binding energy modeling system 100 can utilize the protein preparation model 304 to analyze heavy atom coordinates of the predicted protein binding structure conformation 302 to determine chemically appropriate positions for the hydrogen atom representations 308 according to molecular standards and/or principles. Moreover, the binding energy modeling system 100 can utilize the protein preparation model to analyze the predicted protein binding structure conformation 302 and assign the electron representations 310 to accurately represent electronic properties of the protein, such as formal charges, partial charges, electron distributions, among others. In some embodiments, the binding energy modeling system 100 can utilize the protein preparation model 304 to apply molecular mechanics forcefields to the predicted protein binding structure conformation 302 to evaluate local binding geometry and non-bonded interactions. Based on utilizing the protein preparation model 304 to apply molecular mechanics forcefields, the binding energy modeling system 100 can determine locations to add the hydrogen atom representations 308 and/or the electron representations 310.

For example, in some embodiments the binding energy modeling system 100 can utilize the protein preparation model 304 to process the predicted protein binding structure conformation 302 by analyzing a chemical environment of each heavy atom representation of the predicted protein binding structure conformation 302 to determine appropriate bond orders, protonation states, as well as formal and/or partial charges. Based on performing this analysis, the binding energy modeling system 100 can determine optimized locations for the hydrogen atom representations 308. Further, the binding energy modeling system 100 can utilize the protein preparation model 304 to determine placement for the electron representations 310 based on determining the formal and/or partial charges from the predicted protein binding structure conformation 302.

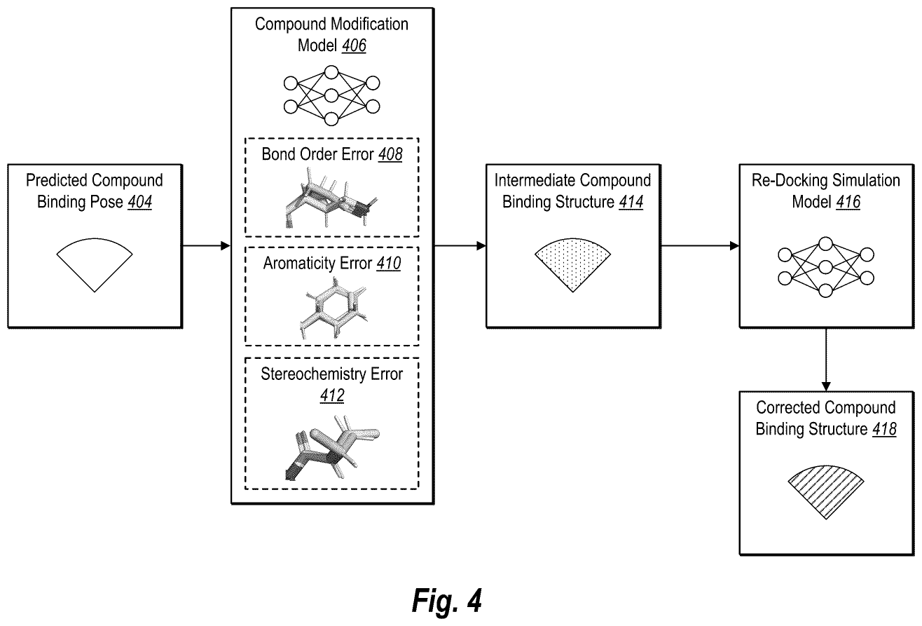

As previously mentioned, the binding energy modeling system 100 can generate a corrected compound binding structure based on a predicted compound binding pose. FIG. 4 illustrates the binding energy modeling system 100 utilizing a compound modification model to identify errors in a predicted compound binding pose and generating a corrected compound binding structure based on identifying the errors in accordance with one or more embodiments.

As shown in FIG. 4 and as previously discussed, the binding energy modeling system 100 can generate a predicted compound binding pose 404 (e.g., utilizing a protein-ligand structural generation machine learning model). The binding energy modeling system 100 can analyze the predicted compound binding pose utilizing a compound modification model 406. As used herein, the term “compound modification model” includes a computer-implemented model (e.g., a machine learning model) that identifies and/or corrects errors in a predicted compound binding pose. For example, the compound modification model can include a machine learning model that the binding energy modeling system 100 trains, finetunes, and/or otherwise utilizes to identify and correct errors in a predicted compound binding pose.

For example, the compound modification model can be a ligand preparation toolkit (e.g., such as OpenEye, OpenEye Spruce Toolkit, OpeneEye OEOmega, QuacPac, Schrödinger LigPrep, RDKit Molecule Prep, MOE Ligand Preparation, ChemAxon Marvin Suite, among others), that the binding energy modeling system 100 utilizes to preprocess the predicted compound binding pose for molecular dynamics modeling. For example, the binding energy modeling system 100 can utilize the compound modification model 406 to optimize proton positions of the predicted compound binding pose 404, to search ligand tautomer states, and/or to assign partial charges to the predicted compound binding pose 404. In some embodiments, the compound modification model 406 can have a same underlying software and/or architecture as the protein preparation model (e.g., the protein preparation model 304 discussed above), and the binding energy modeling system 100 can prompt the underlying software to be the compound modification model 406 by prompting the underlying software to analyze the predicted compound binding pose 404 to generate a chemically valid compound representation (e.g., as opposed to prompting the underlying software to analyze the predicted protein binding structure conformation and generate the corrected protein binding structure conformation).

As shown in FIG. 4, the binding energy modeling system 100 can utilize the compound modification model 406 to identify errors in the predicted compound binding pose. Specifically, the binding energy modeling system 100 can identify the errors by identifying one or more of: a bond order error 408, an aromaticity error 410, or a stereochemistry error 412. As used herein, the term “bond order error” can refer to an incorrect order or arrangement of bonds (e.g., an error in a representation of atomic connectivity) within the predicted compound binding pose. For example, the binding energy modeling system 100 can identify a bond order error by determining a missing bond representation between two atoms, an extraneous bond representation between two atom representations, an incorrect connection or arrangement in order between bonds, an incorrect bond representation type between two atom representations (e.g., such as determining that a bond representation corresponds to a single bond instead of a double bond), and/or an incorrect bond representation length between two atom representations (e.g., such as by determining an error in one or more coordinates for an atom representation that subsequently affects a bond length to a neighboring atom representation.

Along these lines, as used herein, the term “aromaticity error” can refer to an incorrect representation of electron delocalization in a functional group of the compound. For example, the binding energy modeling system 100 can identify an aromaticity error in the predicted compound binding pose by identifying a misrepresented ring structure (e.g., such as a misrepresented benzene ring or a misrepresented heteroatomic ring), such as by determining an extra or a missing bond within a ring structure that prevents delocalization of electron representations within the ring structure.

Further, as used herein, the term “stereochemistry error” can refer to a misrepresentation of stereochemistry within the predicted compound binding pose. To illustrate, the binding energy modeling system 100 can identify the stereochemistry error by determining that, while the predicted compound binding pose accurately depicts connectivity of the atoms of the compound, that there is an error in the spatial orientation of one or more components of the predicted compound binding pose. For example, the binding energy modeling system 100 can identify the stereochemistry error by determining that the predicted compound binding pose depicts an incorrect stereoisomer of the compound, such as an enantiomer and/or a diastereomer of the compound.

As shown in FIG. 4, the binding energy modeling system 100 can utilize the compound modification model 406 to generate an intermediate compound binding structure 414. As used herein, the term “intermediate compound binding structure” can refer to a representation of the compound that the binding energy modeling system 100 generates in response to identifying an error in the predicted compound binding pose. For example, based on identifying the error in the predicted compound binding pose (e.g., such as the bond order error, the aromaticity error, and/or the stereochemistry error), the binding energy modeling system 100 can generate the intermediate compound binding structure to correct the error in the predicted compound binding pose.

To provide an example, the binding energy modeling system 100 can utilize the compound modification model 406 to analyze the predicted compound binding pose 404 and identify the bond order error 408 by determining a double bond representation that should be a single bond representation. The binding energy modeling system 100 can utilize the compound modification model 406 to generate the intermediate compound binding structure 414 by modifying the double bond representation to be a single bond representation. In some embodiments, the binding energy modeling system 100 further modifies one or more coordinates of one or more atomic representations that corresponds to the bond order error.

As another example, the binding energy modeling system 100 can utilize the compound modification model 406 to analyze the predicted compound binding pose 404 and identify the aromaticity error 410 by identifying a missing benzene ring representation within the predicted compound binding pose 404. The binding energy modeling system 100 can utilize the compound modification model 406 to generate the intermediate compound binding structure 414 by modifying the predicted compound binding pose 404 to include a benzene ring representation.

As an additional example, the binding energy modeling system 100 can utilize the compound modification model 406 to analyze the predicted compound binding pose 404 and identify the stereochemistry error 412 by determining that the predicted compound binding pose 404 depicts an incorrect stereoisomer of the compound, such as an enantiomer. Accordingly, the binding energy modeling system 100 can generate the intermediate compound binding structure 414 by generating a correct stereoisomer of the compound.

Further, in some embodiments the binding energy modeling system 100 can identify multiple errors in the predicted compound binding pose 404 (e.g., by identifying one or more of the bond order error 408, the aromaticity error 410, or the stereochemistry error 412). The binding energy modeling system 100 can generate the intermediate compound binding structure 414 by correcting the multiple errors in the predicted compound binding pose 404.

As shown in FIG. 4, the binding energy modeling system 100 can provide the intermediate compound binding structure 414 to a re-docking simulation model 416. As used herein, the term “re-docking simulation model” can refer to a computer-implemented model (e.g., machine learning model) to simulate molecular interactions. For instance, the re-docking simulation model can include a machine learning model that the binding energy modeling system 100 trains, finetunes, or otherwise uses to simulate molecular interactions. For example, the binding energy modeling system 100 can use the re-docking simulation model to simulate the protein and compound binding by docking the intermediate compound binding structure to a representation of the protein (e.g., such as the predicted protein binding structure conformation and/or the corrected protein binding structure conformation).

In some embodiments, the re-docking simulation model 416 can be a ligand docking toolkit (e.g., such as OpenEye Posit and/or OpenEye Hybrid) that the binding energy modeling system 100 utilizes to predict the orientation of a compound representation (e.g., such as the predicted compound binding pose 404, the intermediate compound binding structure 414, and/or a corrected compound binding structure 418) in a simulation of a binding reaction with a representation of a protein (e.g., such as the predicted protein binding structure conformation 302 of FIG. 3 and/or the corrected protein binding structure conformation 306 of FIG. 3). In some embodiments, the binding energy modeling system 100 utilizes the re-docking simulation model 416 to align an initial compound representation (e.g., such as the predicted compound binding pose 404) with a template representation (e.g., such as the predicted protein binding structure conformation 302 of FIG. 3) utilizing a Maximum Common Substructure (MCS) overlay. Further, the binding energy modeling system 100 can utilize the re-docking simulation model 416 to apply shape and pharmacore-based fitting (e.g., to generate pharmacophore queries) and optimization to the predicted compound binding pose 404.

Accordingly, in some embodiments the binding energy modeling system 100 utilizes the re-docking simulation model 416 to identify one or more of the bond order error 408, the aromaticity error 410, and/or the stereochemistry error 412 by docking the predicted compound binding pose 404 to the predicted protein binding structure conformation, and identifying the bond order error 408, the aromaticity error 410, and/or the stereochemistry error 412 from the shape and pharmacore-based fitting.

Further, in some embodiments, based on determining that the intermediate compound binding structure 414 is the corrected compound binding structure 418, the binding energy modeling system 100 can re-equilibrate and/or relax the corrected compound binding structure 418 prior to performing other simulations with the corrected compound binding structure 418. For example, the binding energy modeling system 100 can re-equilibrate the corrected compound binding structure 418 by restoring partial charges, reverting an orientation, and/or modifying structural elements of the corrected compound binding structure to enable the binding energy modeling system 100 to perform a molecular dynamics simulation utilizing the corrected compound binding structure.

To provide an example, the binding energy modeling system 100 can generate the intermediate compound binding structure 414 based on identifying and correcting the bond order error 408 in the predicted compound binding pose 404. Further, the binding energy modeling system 100 can determine that, due to the bond order error 408 of the predicted compound binding pose, the binding energy modeling system 100 generated an inaccurate binding pose between the predicted protein binding structure conformation and the predicted compound binding pose (e.g., such as by determining that the binding energy modeling system 100 could not pair or otherwise dock the predicted compound binding pose to the predicted protein binding structure conformation). The binding energy modeling system 100 can generate the intermediate compound binding structure 414 by correcting the bond order error 408. Further, the binding energy modeling system 100 can provide the intermediate compound binding structure 414 to the re-docking simulation model 416 and can utilize the re-docking simulation model 416 to dock the intermediate compound binding structure 414 to the predicted protein binding structure conformation (or the corrected protein binding structure conformation). Based on successfully utilizing the re-docking simulation model 416 to pair the intermediate compound binding structure with the predicted protein binding structure conformation (or the corrected protein binding structure conformation), the binding energy modeling system 100 can determine that the intermediate compound binding structure is the corrected compound binding structure 418.

In some embodiments, the binding energy modeling system 100 can generate multiple intermediate compound binding structures that correspond to multiple solutions to an error in the predicted compound binding pose. The binding energy modeling system 100 can provide the multiple intermediate compound binding structures to the re-docking simulation model 416 to identify the corrected compound binding structure 418 from the multiple intermediate compound binding structures.

In some embodiments, the binding energy modeling system 100 generates the corrected compound binding structure 418 and the corrected protein binding structure conformation (e.g., the corrected protein binding structure conformation 306 of FIG. 3) to be an OpenEye Design Unit (DU) according to OpenEye parameters for inner and outer contour shapes. Further, the binding energy modeling system 100 can modify formats of the corrected compound binding structure 418 and the corrected protein binding structure conformation to an AMBER format and parameterize the corrected protein binding structure conformation and the corrected compound binding structure 418 using BioSimSpace. Specifically, the binding energy modeling system 100 can parameterize the corrected protein binding structure conformation and the corrected compound binding structure 418 for use by a molecular dynamics simulation model utilizing respective force field models. For example, the binding energy modeling system 100 can parameterize the corrected protein binding structure conformation utilizing AMBER ff14SB. Moreover, the binding energy modeling system 100 can parameterize the corrected compound binding structure 418 utilizing GAFF2.

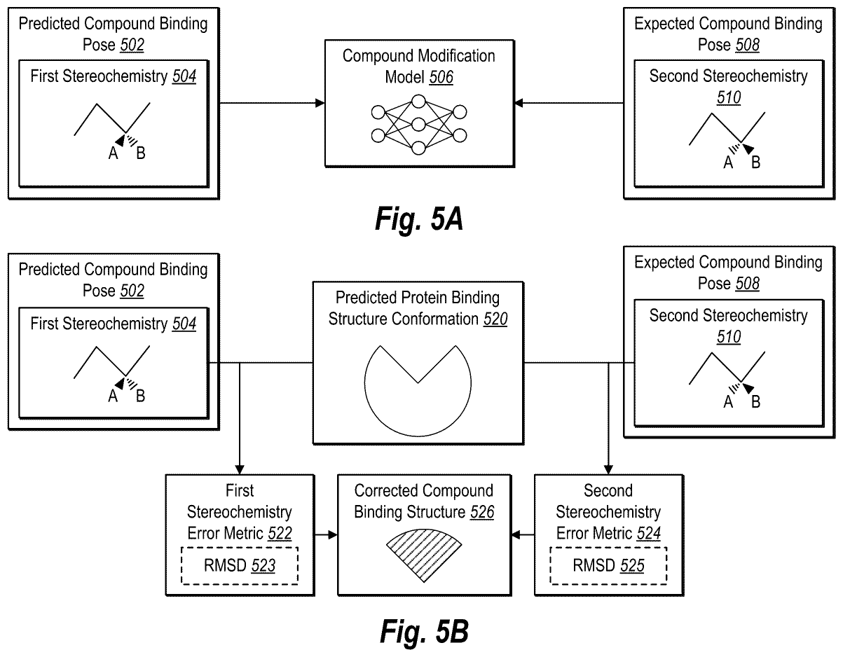

As previously mentioned, the binding energy modeling system 100 can generate a corrected compound binding structure from a predicted compound binding pose by determining a stereochemistry for a predicted compound binding pose. FIGS. 5A-5B illustrate the binding energy modeling system 100 generating compound binding poses having different stereochemistries and determining a corrected compound binding structure based on error metrics for the different stereochemistries in accordance with one or more embodiments.

As shown in FIG. 5A and as previously discussed, the binding energy modeling system 100 can generate (e.g., utilizing a protein-ligand structural generation machine learning model) a predicted compound binding pose 502 for a compound. Indeed, the binding energy modeling system 100 can generate the predicted compound binding pose 502 to include a first stereochemistry 504. For example, the binding energy modeling system 100 can generate the predicted compound binding pose 502 to have the first stereochemistry 504 by determining that a compound input to the protein-ligand structural generation machine learning model indicates a specific enantiomer of the compound (e.g., such as an R enantiomer or an S enantiomer). In some embodiments, the binding energy modeling system 100 determines that the compound input does not indicate a specific stereochemistry and can autonomously determine the first stereochemistry 504.

As illustrated in FIG. 5A, the binding energy modeling system 100 can utilize a compound modification model 506 to compare the predicted compound binding pose with an expected compound binding pose 508 that has a second stereochemistry 510. For example, the binding energy modeling system 100 can determine the expected compound binding pose 508 according to an input to a protein-ligand structural generation machine learning model (e.g., such as a SMILES string and/or a pharmacophore query, among others).

Indeed, the binding energy modeling system 100 can utilize the compound modification model 506 to compare the predicted compound binding pose 502 with the expected compound binding pose 508. For example, the binding energy modeling system 100 can utilize the compound modification model 506 to compare the first stereochemistry 504 with the second stereochemistry 510 to determine that the first stereochemistry 504 is different from the second stereochemistry 510. Further, the binding energy modeling system 100 can utilize the compound modification model 506 to compare different types of compound representations to generate a corrected compound binding structure. For example, the binding energy modeling system 100 can utilize the compound modification model 506 to compare an output of a protein-ligand structural generation machine learning model with an input of the protein-ligand structural generation machine learning model to determine compound stereochemistry representation discrepancies between inputs and outputs of the protein-ligand structural generation machine learning model.

To illustrate, the binding energy modeling system 100 can utilize the compound modification model 506 to compare the first stereochemistry 504 and the second stereochemistry 510 to determine that the first stereochemistry 504 and the second stereochemistry 510 represent different spatial representations of the compound. Indeed, the binding energy modeling system 100 can compare the predicted compound binding pose 502 and the expected compound binding pose 508 to determine that each respective stereochemistry includes the same connectivity of molecular representations, but different spatial orientations. In some embodiments, the binding energy modeling system 100 determines that the first stereochemistry 504 and the second stereochemistry 510 are different spatial representations of a functional group of the compound. In some embodiments, the binding energy modeling system 100 determines that the first stereochemistry 504 and the second stereochemistry 510 represent different molecular spatial representations (e.g., such as different versions of the same compound that have axial chirality, planar chirality, and/or helical chirality, among others). Based on utilizing the compound modification model 506 to determine that the first stereochemistry 504 is different from the second stereochemistry 510, the binding energy modeling system 100 can compare the predicted compound binding pose 502 and the expected compound binding pose 508 to a predicted protein binding structure conformation (and/or a corrected protein binding structure conformation) to determine respective stereochemistry error metrics for each respective stereochemistry.

As shown in FIG. 5B, the binding energy modeling system 100 can compare the predicted compound binding pose 502 to a predicted protein binding structure conformation 520 to determine a first stereochemistry error metric 522 based on the first stereochemistry 504. As used herein, the term “stereochemistry error metric” refers to a measure of statistical similarity of an overlap between a representation of a compound and a representation of a protein within a representation of a binding interaction. For example, the binding energy modeling system 100 can generate a stereochemistry error metric by determining how a stereochemistry representation (e.g., of a functional group or a molecule) affects a binding of a compound representation and a protein representation. For example, the binding energy modeling system 100 can generate the stereochemistry error metric to quantify overlapping surface areas of a protein representation and a compound representation. Moreover, the binding energy modeling system 100 can generate the stereochemistry error to represent a level of deviation between a subset of the protein representation and a subset of the compound representation (e.g., a level of deviation between 3D coordinates for the subset of the protein representation and 3D coordinates for the subset of the compound representation).

Indeed, as shown in FIG. 5B, the binding energy modeling system 100 can generate the first stereochemistry error metric 522 based on comparing the first stereochemistry 504 of the predicted compound binding pose 502 to the predicted protein binding structure conformation 520. For example, the binding energy modeling system 100 can simulate docking the predicted compound binding pose 502 to the predicted protein binding structure conformation 520 (e.g., such as using a re-docking simulation model) to determine how the first stereochemistry 504 impacts the docking. The binding energy modeling system 100 can further determine a first set of coordinates for atomic representations of the predicted protein binding structure conformation 520 and a second set of coordinates for atomic representations of the predicted compound binding pose 502. The binding energy modeling system 100 can determine the first stereochemistry error metric 522 by comparing the first set of coordinates and the second set of coordinates to determine a disparity between the sets of coordinates. By determining the disparity between the sets of coordinates, the binding energy modeling system 100 can quantify a spatial proximity of the predicted compound binding pose 502 to the predicted protein binding structure conformation 520. In some embodiments, the binding energy modeling system 100 determines the first stereochemistry error metric 522 by determining a root mean square deviation (RMSD) 523 between the predicted compound binding pose 502 and the predicted protein binding structure conformation 520. To illustrate, the binding energy modeling system 100 can determine the RMSD 523 between the first set of coordinates and the second set of coordinates.

As illustrated in FIG. 5B, the binding energy modeling system 100 can compare the predicted protein binding structure conformation 520 to the second stereochemistry 510 of the expected compound binding pose 508 (and/or a representation of the expected compound binding pose 508 that the binding energy modeling system 100 generates to simulate docking the expected compound binding pose 508 to the predicted protein binding structure conformation 520) to determine a second stereochemistry error metric 524. Indeed, the binding energy modeling system 100 can simulate docking the expected compound binding pose 508 to the predicted protein binding structure conformation 520 (e.g., such as using a re-docking simulation model) to determine how the second stereochemistry 510 impacts the docking. In some embodiments, the binding energy modeling system 100 determines the second stereochemistry error metric 524 based at least in part on the expected compound binding pose 508.

For example, the binding energy modeling system 100 can determine a third set of coordinates for atomic representations of the expected compound binding pose 508. The binding energy modeling system 100 can determine the second stereochemistry error metric 524 by comparing the first set of coordinates (e.g., of the predicted protein binding structure conformation 520) to the third set of coordinates to determine a disparity between the sets of coordinates. By determining the disparity between the sets of coordinates, the binding energy modeling system 100 can quantify a proximity of the expected compound binding pose 508 to the predicted protein binding structure conformation 520. Additionally, in some embodiments the binding energy modeling system 100 determines the second stereochemistry error metric 524 by determining an RMSD 525 between the expected compound binding pose 508 and the predicted protein binding structure conformation 520. For example, the binding energy modeling system 100 can determine the RMSD 525 between the first set of coordinates and the third set of coordinates.

As shown in FIG. 5B, the binding energy modeling system 100 can compare the first stereochemistry error metric 522 and the second stereochemistry error metric 524 to identify a corrected compound binding structure 526. For example, the binding energy modeling system 100 can compare the first stereochemistry error metric 522 and the second stereochemistry error metric to determine which stereochemistry error metric indicates a higher (comparative) level of proximity between the respective compound representation and the predicted protein binding structure conformation 520. For example, where the first stereochemistry error metric 522 is the RMSD 523 and the second stereochemistry error metric is the RMSD 523, the binding energy modeling system 100 can determine the corrected compound binding structure 526 by selecting a compound binding pose (between the predicted compound binding pose 502 and the expected compound binding pose 508) that corresponds to a lower RMSD.

To illustrate, the binding energy modeling system 100 can compare the first stereochemistry error metric 522 and the second stereochemistry error metric 524 to determine that the RMSD 525 is lower than the RMSD 523. Based on determining that the RMSD 525 is lower than the RMSD 523, the binding energy modeling system 100 can select the expected compound binding pose 508 to be the corrected compound binding structure 526. Further, based on determining that the RMSD 525 is lower than the RMSD 523, the binding energy modeling system 100 can determine that selecting the second stereochemistry 510 enables a more accurate simulation of the binding reaction between the protein and the compound.

In some embodiments, the binding energy modeling system 100 generates the first stereochemistry error metric 522 and the second stereochemistry error metric 524 to be a statistical metric other than an RMSD. To illustrate, the binding energy modeling system 100 can generate the first stereochemistry error metric 522 and the second stereochemistry error metric 524 to be a Pearson's correlation coefficient, a mean unsigned error metric, or a Kendall's Tau metric, among others.

Further, in some embodiments the binding energy modeling system 100 compares the predicted compound binding pose 502 and/or the expected compound binding pose 508 to a predicted protein binding structure conformation (and/or a corrected protein binding structure conformation) to determine the first stereochemistry error metric 522 and/or the second stereochemistry error metric 524. Moreover, the binding energy modeling system 100 can generate multiple expected compound binding poses that have different stereochemistries and can compare the multiple expected compound binding poses to the predicted protein binding structure conformation 520 and/or to the corrected protein binding structure conformation to determine stereochemistry errors for each expected compound binding pose.

Moreover, while the previous discussion of FIGS. 5A-5B relates to determining how different stereochemistries influence binding simulations between protein representations and compound representations, the binding energy modeling system 100 can compare protein representations (e.g., such as the predicted protein binding structure conformation 520 or a corrected protein binding structure conformation) to compound representations (e.g., such as the predicted compound binding pose 502, an intermediate compound binding structure, the expected compound binding pose 508, or a corrected compound binding structure) to determine overlap metrics that indicate a proximity and/or a goodness of fit protein representation with the compound representation. For example, the binding energy modeling system 100 can compare an intermediate compound binding structure that the binding energy modeling system 100 generates in response to determining a bond order error and/or an aromaticity error, to the predicted protein binding structure conformation 520 (and/or to a corrected protein binding structure conformation) to generate an overlap metric. Based on generating the overlap metric, the binding energy modeling system 100 can determine to generate an additional intermediate compound binding structure, or the binding energy modeling system 100 can select the intermediate compound binding structure to be the corrected compound binding structure 526.

In some embodiments, the binding energy modeling system 100 utilizes the compound modification model 506 (and/or a protein-ligand structural generation model) to generate multiple predicted compound binding poses, where each of the multiple predicted compound binding poses includes a different stereochemistry. To illustrate, the binding energy modeling system 100 can determine that an input to the protein-ligand structural generation machine learning model does not specify any stereochemistry for the compound and/or that the input to the protein-ligand structural generation machine learning model indicates multiple possible stereochemistries for the compound. Based on generating the multiple predicted compound binding poses corresponding to the multiple stereochemistries, the binding energy modeling system 100 can further compare (e.g., utilizing the compound modification model and/or a re-docking simulation model) each of the multiple predicted compound binding poses to the predicted protein binding structure conformation 520 (and/or a corrected protein binding structure conformation) to determine corresponding stereochemistry error metrics. The binding energy modeling system 100 can further determine the corrected compound binding structure 526 according to the corresponding stereochemistry error metrics.

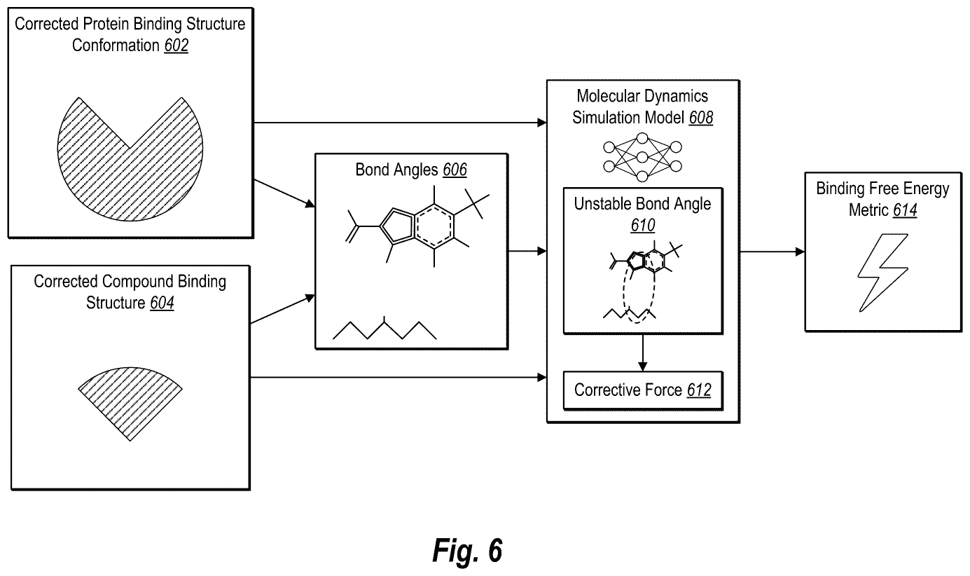

As previously mentioned, the binding energy modeling system 100 can utilize a molecular dynamics simulation model to generate a binding free energy metric. FIG. 6 illustrates the binding energy modeling system 100 generating a binding free energy metric based on utilizing a molecular dynamics simulation model to apply a corrective force to correct an unstable bond angle in accordance with one or more embodiments.

As previously discussed, the binding energy modeling system 100 can utilize a molecular dynamics simulation model 608 to simulate a binding interaction between a protein representation (e.g., such as a corrected protein binding structure conformation 602) and a compound representation (e.g., such as a corrected compound binding structure 604) to determine a binding free energy metric 614. To determine the binding free energy metric 614, the binding energy modeling system 100 can utilize the molecular dynamics simulation model 608 to manipulate the corrected protein binding structure conformation 602 and the corrected compound binding structure 604 within a three-dimensional space over time. Specifically, the binding energy modeling system 100 can utilize the molecular dynamics simulation model 608 to apply one or more force field functions to the corrected protein binding structure conformation 602 and the corrected compound binding structure 604 that represent how atoms/molecules/functional groups of the corrected protein binding structure conformation 602 and the corrected compound binding structure 604 are attracted to each other. For example, the binding energy modeling system 100 can determine the one or more force field functions based on molecular properties, bond lengths, bond angles, and/or chemical properties that correspond to the protein that the corrected protein binding structure conformation 602 represents and the compound that the corrected compound binding structure 604 represents.

Specifically the binding energy modeling system 100 can utilize the molecular dynamics simulation model 608 to evaluate binding stability by performing a series of controlled simulations according to a λ schedule. Specifically, the binding energy modeling system 100 can generate the λ schedule to define a series of λ windows, wherein each λ window corresponds to a controlled simulation of the series of controlled simulations and defines a different interaction strength between the corrected protein binding structure conformation 602 and the corrected compound binding structure 604. Indeed, the binding energy modeling system 100 can utilize the molecular dynamics simulation model 608 to progress through the λ schedule, starting with the corrected protein binding structure conformation 602 and the corrected compound binding structure 604 in a bound state and gradually decreasing the interaction strength for each λ window to transition the corrected protein binding structure conformation 602 and the corrected compound binding structure 604 into respective unbound states.

Further, while the binding energy modeling system 100 utilizes the molecular dynamics simulation model 608 to perform the series of controlled interactions, the binding energy modeling system 100 can determine a first set of restraint atom representations that corresponds to the corrected protein binding structure conformation 602 and a second set of restraint atom representations that corresponds to the corrected compound binding structure 604. Specifically, the binding energy modeling system 100 can utilize the first set of restraint atoms and the second set of restraint atoms to constrain the position and rotation of the corrected protein binding structure conformation 602 and the corrected compound binding structure 604, respectively, to keep each structure representation in a fixed orientation and to prevent rotation during execution of the λ schedule. Further, the binding energy modeling system 100 can apply bond angle stability constraints to the first set of restraint atoms and/or to the second set of restraint atoms to ensure geometric stability in the simulations. Specifically, the binding energy modeling system 100 can apply the bond angle stability constraints to prevent bond angles of the first set of restraint atoms and/or the second set of restraint atoms from reaching geometrically unstable angles (e.g., such as 0° or 180°) that can cause computational instability in molecular dynamics simulations. Indeed, based at least in part on applying the bond angle stability constraints to the first and/or second set of restraint atoms, the binding energy modeling system 100 can generate and execute the λ schedule to incrementally and accurately determine changes in the binding free energy. For instance, in some implementations, the binding energy modeling system 100 utilizes the approach described in Provisional Application No. 63/773,745, entitled “Optimizing Absolute Binding Free Energy Calculations for Production Usage,” filed Mar. 18, 2025, the contents of which are incorporated by reference herein in their entirety.

As shown in FIG. 6 and as previously discussed, the binding energy modeling system 100 can generate a corrected protein binding structure conformation 602 and a corrected compound binding structure 604. Based on generating the corrected protein binding structure conformation 602 and the corrected compound binding structure 604, the binding energy modeling system 100 can determine bond angles 606 from the corrected protein binding structure conformation 602 and/or the corrected compound binding structure 604. As used herein, the term “bond angle” can refer to a geometric measurement that defines an arrangement between components between (or within) a protein representation and/or a compound representation. To illustrate, a bond angle can be a geometric measurement that describes a planar relationship between molecular representations and/or groups of molecular representations (e.g., such as atoms, functional groups, or side chains, among others) in a protein representation and/or a compound representation. In some embodiments, the bond angles can be geometric measurements between one or more components of a protein representation and one or more components of a compound representation. For example, a bond angle can describe a relationship between components of a compound and of a protein in a bound conformation (e.g., such as a binding simulation) between the compound representation and the protein representation.

In some embodiments, the binding energy modeling system 100 determines the bond angles 606 to correspond to a first set of restraint atom representations for the corrected protein binding structure conformation 602 and a second set of restraint atom representations for the corrected compound binding structure 604. For example, the binding energy modeling system 100 can determine a first set of bond angles that correspond to the first set of restraint atom representations and a second set of bond angles that correspond to the second set of restraint atom representations. The binding energy modeling system 100 can determine the bond angles 606 to be the first set of bond angles and/or the second set of bond angles. The binding energy modeling system 100 can determine the bond angles 606 utilizing a machine learning model, such as a protein-ligand structural generation machine learning model, a protein preparation model, or a compound modification model, among others. For example, the binding energy modeling system 100 can determine the first set of bond angles utilizing the protein preparation model and the second set of bond angles utilizing the compound modification model. Moreover, the binding energy modeling system 100 can determine the bonding angles at different iterations of applying the molecular dynamics simulation model 608.

As shown in FIG. 6, the binding energy modeling system 100 can provide the corrected protein binding structure conformation 602, the corrected compound binding structure 604, and the bond angles 606 to a molecular dynamics simulation model 608. In some embodiments the binding energy modeling system 100 provides the bond angles 606 to the molecular dynamics simulation model 608 by specifically annotating or otherwise demarcating the first set of restraint atoms and the corresponding first set of bond angles for the corrected protein binding structure conformation 602, as well as the second set of restraint atoms and the corresponding second set of bond angles for the corrected compound binding structure 604.

As used herein, the term “molecular dynamics simulation model” refers to a machine learning model that the binding energy modeling system 100 utilizes to simulate biological interactions (e.g., such as a molecular dynamics simulation). For example, the binding energy modeling system 100 can provide chemically and/or biologically valid representations of a protein and a compound (e.g., such as a corrected protein binding structure conformation and a corrected compound binding structure) to the molecular dynamics simulation model and can utilize the molecular dynamics simulation model to simulate or otherwise model a biological interaction, such as a binding interaction, between the corrected protein binding structure conformation and the corrected compound binding structure. For instance, a molecular dynamics model includes a computational model that simulates physical motions of atoms and molecules over time by solving equations of motion for a system of interacting particles. Thus, a molecular dynamics model determines how molecules move, vibrate, and interact according to physics. A molecular dynamics model can utilize statistical mechanics to estimate the free-energy difference between thermodynamic states by simulating reversible transformations between them. Molecular dynamics models include Amber, GROMACS, NAMD, Open MM, or Schrodinger FEP+.

Indeed, as opposed to a protein-ligand structural generation machine learning model (e.g., a co-folding model such as Boltz or Boltz-2), the binding energy modeling system 100 can utilize the molecular dynamics simulation model to generate or otherwise determine energetic properties of chemically complete representations of proteins and compounds in a molecular dynamics reaction. For example, the binding energy modeling system 100 can utilize the protein-ligand structural generation machine learning model to generate co-dependent 3D spatial representations of a protein and a compound. The binding energy modeling system 100 can augment the 3D spatial representations to generate chemically valid 3D spatial representations of the protein and the compound. The binding energy modeling system 100 can utilize the molecular dynamics simulation model to compute atomic forces and energy gradients between the chemically valid 3D spatial representations of the protein and the compound (e.g., the corrected protein binding structure conformation and the corrected compound binding structure). Indeed, whereas the binding energy modeling system 100 utilizes a protein-ligand structural generation machine learning model to determine statistically likely co-dependent conformations for a protein and a compound (e.g., such as a predicted protein binding structure conformation and a predicted compound binding pose), the binding energy modeling system 100 can utilize the protein-ligand structural generation machine learning model to model the molecular dynamics forces (e.g., such as the physics) that drive or otherwise create the binding conformation representations for the protein and the compound.

Moreover, the molecular dynamics simulation model 608 can be a physics-informed neural network that the binding energy modeling system 100 utilizes to predict potential energies, atomic forces, and/or time-dependent trajectories. Indeed, the molecular dynamics simulation model can include a differentiable molecular dynamics engine. In some embodiments, the molecular dynamics simulation model 608 includes a molecular mechanics engine that the binding energy modeling system 100 utilizes to calculate binding energies from time-resolved atomic interactions. For example, the molecular dynamics simulation model 608 can be a free-energy perturbation (FEP) model, a machine-learning augmented molecular dynamics model (e.g., such as TorchMD-Net, ANI, or DeepMD), or a hybrid quantum mechanics/molecular mechanics (QM/MM) model).

Thus, a protein-ligand structural generation machine learning model can include a deep neural network that applies statistical learning from data to generate predicted 3D structures, binding poses, and/or free energies (directly from inference). In contrast, the molecular dynamics simulation model can simulate Newtonian physics through deterministic integration of equations of motion (from force field parameters derived from physics and quantum mechanics) to generate atomic trajectories over time (and thermodynamic quantities). The protein-ligand structural generation machine learning model (in some implementations) can take seconds to minutes to run, whereas molecular dynamics simulation models can take hours to weeks to run. By utilizing a protein-ligand structural generation machine learning model, correcting resulting proteins and compounds, and then utilizing a molecular dynamics simulation model, the binding energy modeling system 100 can significantly improve the resulting predictions as well as save resources (by reducing implementation of the molecular dynamics simulation model).

For example, the binding energy modeling system 100 can simulate the biological interaction between the corrected protein binding structure conformation 602 and the corrected compound binding structure 604 by utilizing the molecular dynamics simulation model 608 to apply a series of controlled simulations of a λ schedule in λ windows as discussed above. For example, the binding energy modeling system 100 can iteratively perform controlled simulations of a λ schedule by decreasing forces of the force field functions at each λ window.

As shown in FIG. 6, the binding energy modeling system 100 can utilize the molecular dynamics simulation model 608 to determine an unstable bond angle 610. As used herein, the term “unstable bond angle” can refer to a bond angle that includes components of a protein representation and components of a compound representation. Specifically, an unstable bond angle can refer to an alignment or orientation of a protein representation and a compound representation that creates computational strain in a simulation of a biological interaction (e.g., such as a molecular dynamics simulation). Indeed, the unstable bond angle can be an angle between a first set of atom representations that corresponds to the protein representation and a second set of atoms that corresponds to the compound representation.

For example, the binding energy modeling system 100 can determine the unstable bond angle 610 from the bond angles 606 as the binding energy modeling system 100 modifies application of the force field functions according to the λ schedule. Indeed, the unstable bond angle 610 can be a bond angle that causes operational or computational errors in a molecular dynamics simulation by creating harmonic instabilities within the molecular dynamics simulation. For example, the unstable bond angle 610 can be a simulation destabilizing angle that destabilizes operations of molecular dynamics simulation, as opposed to an angle that creates chemical strain within the molecular dynamics situation (e.g., such as by creating opposing forces between a functional group representations in protein and compound representations).

In some embodiments, the binding energy modeling system 100 determines the unstable bond angle to be angles that approach 0° or 180° (e.g., the binding energy modeling system 100 can determine that alignments or conformations between components of the compound representation and the protein representation that approach 0° or 180° will destabilize the molecular dynamics simulation). Based on identifying the unstable bond angle 610, the binding energy modeling system 100 can utilize the molecular dynamics simulation model 608 to apply a corrective force (e.g., such as a corrective force field function) to constituent components of the unstable bond angle 610 (e.g., such as atomic, molecular, and/or functional group representations) to prevent the unstable bond angle 610 from reaching 0° or 180°. In some embodiments, the binding energy modeling system 100 applies a gradient-based corrective force and scales the magnitude of the gradient-based corrective force in response to an increasing risk of a simulation failure due to the unstable bond angle 610 approaching 0° or 180°. For example, the binding energy modeling system 100 can monitor the unstable bond angle 610 through the λ schedule to determine how the unstable bond angle changes within each λ window. The binding energy modeling system 100 can scale the corrective force 612 accordingly, such that as the unstable bond angle 610 becomes closer to 0° or 180°, the binding energy modeling system 100 can accordingly increase the corrective force 612 to force constituents of the unstable bond angle to an angle that does not pose a risk of simulation failure.

In some embodiments, the binding energy modeling system 100 utilizes the molecular dynamics simulation model 608 to apply bond angle stability constraints to any bond angle of a set of restraint atoms (e.g., restraint atom representations) that approaches 0° or 180°. For example, the binding energy modeling system 100 can monitor the bond angles 606 and apply the bond angle stability constraints to any bond angle of the bond angles 606 that approaches 0° or 180°. Indeed, the binding energy modeling system 100 can train or otherwise finetune the molecular dynamics simulation model 608 to implement the bond angle stability constraints by implementing the corrective force 612.

As illustrated in FIG. 6, the binding energy modeling system 100 can utilize the molecular dynamics simulation model 608 to generate the binding free energy metric 614. Specifically, the binding energy modeling system 100 can generate the binding free energy metric 614 to be an absolute binding free energy metric (or another binding energy metric) from the binding simulation between the corrected protein binding structure conformation 602 and the corrected compound binding structure 604. In some embodiments, the binding energy modeling system 100 determines the binding free energy metric 614 by determining a difference in energy levels for the protein representation and the compound representation in a bound and in an unbound state. Specifically, the binding energy modeling system 100 can determine the binding free energy metric 614 from a summation of values from each λ window of the λ schedule.

Further, in some embodiments the binding energy modeling system 100 assigns molecular dynamics simulation parameters to the corrected compound binding structure 604 for the molecular dynamics simulation utilizing the molecular dynamics simulation model 608 or a compound modification model (e.g., the compound modification model 406 of FIG. 4). For example, the binding energy modeling system 100 can utilize OpenFF 2.2.1 to assign the molecular dynamics simulation parameters.

Moreover, in some embodiments the binding energy modeling system 100 utilizes the molecular dynamics simulation model 608 to simulate solvation of a protein-ligand complex (e.g., solvation of a bound conformation of the corrected protein binding structure conformation 602 and the corrected compound binding structure 604). For example, the binding energy modeling system 100 can utilize a TIP3P water model within a rhombic dodecahedron simulation box to simulate solvation of the bound conformation, maintaining a shell distance of 0.5 nm between the solute and its periodic neighbor. Further, the binding energy modeling system 100 can neutralize the solvent simulation with a concentration representation of NaCL at a concentration of 0.15M.

In some embodiments, such as where the binding energy modeling system 100 generates the corrected compound binding structure 604 to represent a charged ligand, the binding energy modeling system 100 can utilize the molecular dynamics simulation model 608 to simulate solvation of the protein-ligand complex in a cubic box and can maintain a shell distance of 0.8 nm. To neutralize simulations involving a charged ligand, the binding energy modeling system 100 can apply alchemical ion restraints to ions positioned at corners of the simulation box.

Additionally, in some embodiments the binding energy modeling system 100 simulates solvation of a representation of an unbound (e.g., free) corrected compound binding structure 604 in a cubic box of dimensions of 4 nm×4 nm×4 nm.

Further, as previously mentioned, the binding energy modeling system 100 can utilize the molecular dynamics simulation model 608 to simulate the molecular dynamics reaction between the corrected protein binding structure conformation 602 and the corrected compound binding structure 604 utilizing a λ schedule that includes λ windows. In some embodiments, such as when the binding energy modeling system 100 generates the corrected compound binding structure 604 to represent an unbound representation of the ligand, the binding energy modeling system 100 generates the λ schedule to include 48λ windows. In some embodiments, such when the binding energy modeling system 100 generates the corrected compound binding structure 604 to represent a bound representation of a charged ligand, the binding energy modeling system 100 generates the λ schedule to include 96λ windows. In some embodiments, such as when the binding energy modeling system 100 generates the corrected compound binding structure 604 to represent a bound representation of a neutral ligand, the binding energy modeling system 100 generates the λ schedule to include 64λ windows. In some embodiments, the binding energy modeling system 100 generates the λ schedule to include a subset of λ windows that corresponds to a bound representation of the ligand and a subset of λ windows that corresponds to an unbound representation of the ligand. Further, the binding energy modeling system 100 can determine the binding free energy metric 614 by utilizing a binding free energy determination model, such as alchemlyb, to aggregate binding free energy metric data for all of the λ windows of the λ schedule.

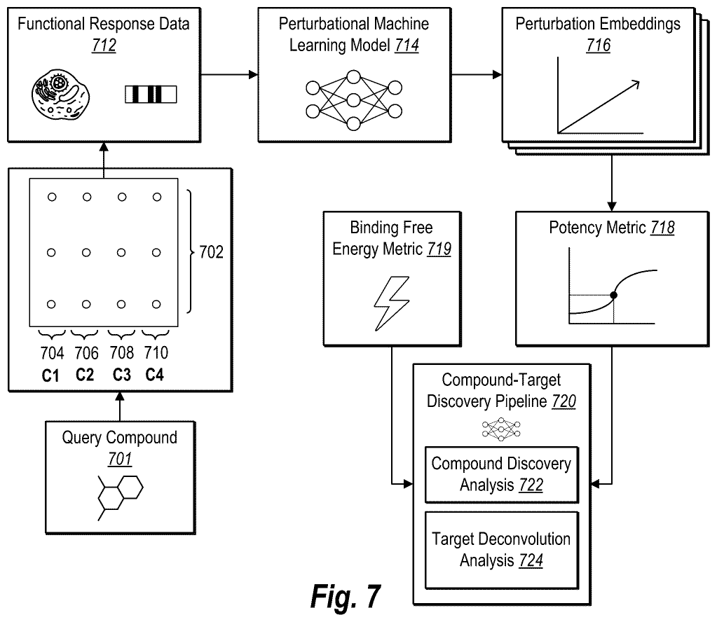

As previously mentioned, in some embodiments the binding energy modeling system 100 determines a potency metric for a compound. FIG. 7 illustrates the binding energy modeling system 100 initiating a compound-target discovery pipeline based on determining a potency metric for the compound in accordance with one or more embodiments.

As shown in FIG. 7, the binding energy modeling system 100 can apply a query compound 701 to a plurality of cells 702 (e.g., such as to perform a compound perturbation). As used herein, the term “query compound” can refer to a chemical compound that is the subject of a query, experiment, or analysis. In some embodiments, the binding energy modeling system 100 selects the query compound 701 to correspond to the compound that the binding energy modeling system 100 generates representations of (e.g., such as a predicted compound binding pose, an expected compound binding pose, an intermediate compound binding structure, and/or a corrected compound binding structure) to determine a free energy binding metric. Further, in some embodiments the binding energy modeling system 100 applies the query compound to the plurality of cells 702 as part of a biochemical assay.

Similarly, as used herein, the term “cell” can refer to a structural, functional, and biological unit of living organisms (e.g., such as a biological cell). Specifically, a cell can vary in size shape, and function depending on the organism and the role of the cell. For example, a cell can include a plasma membrane to separate the internal cell environment from the external surroundings and the cell can further contain genetic material.

As shown in FIG. 7, the binding energy modeling system 100 can apply the query compound 701 to the plurality of cells 702 in a plurality of concentrations 704, 706, 708, 710. For example, the plurality of cells 702 can be arranged in columns of wells on a plate. The binding energy modeling system 100 can apply a first concentration 704 (e.g., “C1”) of the query compound 701 to a first column of the plate, a second concentration 706 (e.g., “C2”), of the query compound 701 to a second column of the plate, a third concentration 708 (e.g., “C3”) of the query compound 701 to a third column of the plate, and a fourth concentration 710 (e.g., “C4”) of the query compound 701 to a fourth column of the plate. The term concentration refers to an amount of a compound in a particular volume (e.g., of a cell or a fluid). Because the volume is often the same or similar across experiments, the disclosure herein uses concentration to also refer to an overall amount (or dose) of compound introduced to a cell (e.g., C1 can refer to a first dose of the compound applied to a cell and C2 can refer to a second dose of compound applied to a cell).

Further, while FIG. 7 illustrates the binding energy modeling system 100 applying the query compound 701 to the plurality of cells 702 on a per-column basis, it will be appreciated that per-column application of the query compound 701 is for illustrative purposes only. Indeed, the binding energy modeling system 100 is not limited to applying the query compound 701 in a per-column basis. The binding energy modeling system 100 can apply the query compound 701 in any suitable manner, such as a per-row basis, random wells, a per-column basis, or any other suitable manner.

As illustrated in FIG. 7, based on applying the query compound 701 in the plurality of concentrations 704, 706, 708, 710 to the plurality of cells 702, the binding energy modeling system 100 can determine functional response data 712 from the plurality of cells 702. As used herein, the term “functional response data” can refer to an impact on a cell (or a plurality of cells) caused by a perturbation, such as an application of the query compound 701. For example, functional response data can include measures of phenotypic changes, gene expression alterations, and/or protein-level effects, among others.

For example, the binding energy modeling system 100 can determine the functional response data 712 for the query compound 701 by capturing or otherwise generating phenomic images of the plurality of cells 702 during and/or after the binding energy modeling system 100 applies the query compound 701 to the plurality of cells. In some embodiments, the binding energy modeling system 100 can determine the functional response data 712 for the query compound 701 by determining transcriptomic profiles for the plurality of cells 702. For example, the binding energy modeling system 100 can determine the transcriptomic profiles (utilizing sequencing machines and sequencing machine learning models) by determining RNA counts (e.g., such as mRNA) for the plurality of cells 702 resulting from exposure to the plurality of concentrations 704, 706, 708, 710 of the query compound 701. In some embodiments the binding energy modeling system 100 determines the functional response data 712 by generating or otherwise capturing phenomic images of the plurality of cells 702 and by determining transcriptomic profiles for the plurality of cells 702.

As shown in FIG. 7, the binding energy modeling system 100 can provide the functional response data 712 for the query compound 701 to a perturbation machine learning model 714. As used herein, the term “perturbation machine learning model” can refer to a machine learning model that the binding energy modeling system 100 utilizes to analyze and generate representations of functional response data (e.g., that the binding energy modeling system 100 utilizes to generate representations of perturbations). For example, the perturbation machine learning model 714 can be a masked autoencoder (MAE), a variational autoencoder (VAE), a contrastive learning model, a transformer, a convolutional neural network, and/or a classifier.

As shown in FIG. 7, based on providing the functional response data 712 to the perturbation machine learning model 714, the binding energy modeling system 100 can use the perturbation machine learning model 714 to generate perturbation embeddings 716. As used herein, the term “perturbation embedding” can refer to a machine learning embedding (e.g., a vector representation in latent machine learning space) of a particular compound concentration. For example, the binding energy modeling system 100 can generate a perturbation embedding by generating a vector in latent feature space that represents functional response data for a particular concentration.

As shown in FIG. 7, based on generating the perturbation embeddings 716, the binding energy modeling system 100 can determine a potency metric 718 for the query compound 701. As used herein, the term “potency metric” can refer to a value that indicates a potency for a compound. For example, the binding energy modeling system 100 can generate the potency metric to represent a correlation between concentration and response or impact.

In some embodiments, the binding energy modeling system 100 generates the potency metric 718 to correspond to a specific concentration of the plurality of concentrations 704, 706, 708, or 710. For example, the binding energy modeling system 100 can generate the potency metric 718 to be a half-maximal effective concentration (e.g., an EC50). In some embodiments, the binding energy modeling system 100 generates the potency metric 718 to be an area under the curve metric. Further, the binding energy modeling system 100 can generate the potency metric 718 to be a combination of potency metrics, such as a combination of the EC50 and the area under the curve metric.

As shown in FIG. 7, based on generating the potency metric 718, the binding energy modeling system 100 can determine to initiate or otherwise modify a compound-target discovery pipeline 720. For example, in some embodiments the binding energy modeling system 100 compares the potency metric 718 to a binding free energy metric 719 and determines to perform additional perturbations and/or assays based on the comparison. In some embodiments, the binding energy modeling system 100 generates the binding free energy metric 719 and subsequently determines the potency metric 718 as a part of the compound-target discovery pipeline 720. In some embodiments, the binding energy modeling system 100 initiates the compound-target discovery pipeline 720 by initiating a compound discovery assay based on the potency metric 718 and/or the binding free energy metric 719.

As shown in FIG. 7, the binding energy modeling system 100 can initiate or otherwise perform the compound-target discovery pipeline 720 by implementing or otherwise performing compound discovery analysis 722 based on determining the binding free energy metric 719, the potency metric 718, and/or comparing the binding free energy metric 719 and the potency metric 718. Further, the binding energy modeling system 100 can initiate or otherwise perform the compound-target discovery pipeline 720 by performing target deconvolution analysis 724. In some embodiments, the binding energy modeling system 100 performs the target deconvolution analysis 724 based on performing the compound discovery analysis 722.

In some embodiments, the binding energy modeling system 100 can initiate the compound-target discovery pipeline 720 by determining a similarity measure between different types of perturbations. For example, the binding energy modeling system 100 can generate the perturbation embeddings 716 based on performing a compound perturbation (e.g., such as applying the query compound 701 to the plurality of cells 702 in the plurality of concentrations 704, 706, 708, 710). Further, the binding energy modeling system 100 can apply a target perturbation (e.g., such as a compound perturbation for a different compound than the query compound 701 or a different type of perturbation, such as a gene-knockout perturbation) to a cell and can utilize the perturbation machine learning model 714 (and/or a different machine learning model) to generate a target perturbation embedding (e.g., such as a vector representation) that represents effects of the target perturbation on the cell.

Further, the binding energy modeling system 100 can compare the target perturbation embedding to the perturbation embeddings 716 to determine a similarity metric (e.g., such as a cosine similarity metric) between the perturbation embeddings 716 and the target perturbation embedding for each concentration of the plurality of concentrations 704, 706, 708, 710. The binding energy modeling system 100 can utilize the similarity metric to determine the potency metric 718. Further, the binding energy modeling system 100 can compare the potency metric 718 to the binding free energy metric to determine additional actions to take (e.g., such as performing additional perturbations and/or performing additional assays, among others). More information regarding the binding energy modeling system 100 comparing perturbation embeddings (e.g., such as compound perturbation embeddings) to target perturbation embeddings to determine potency metrics (e.g., such as concentration-target embedding similarity measures) can be found in U.S. patent application Ser. No. 19/331,878, titled “UTILIZING COMPOUND PERTURBATION CONCENTRATION MACHINE LEARNING EMBEDDINGS AND TARGET PERTURBATION MACHINE LEARNING EMBEDDINGS TO GENERATE POTENCY METRICS FOR IMPROVED COMPOUND DISCOVERY,” filed Sep. 17, 2025, which is incorporated by reference herein in its entirety.

To further illustrate, the binding energy modeling system 100 can initiate or modify the compound-target discovery pipeline 720 to perform one or more of the following tasks: target identification, hit identification, hit-to-lead optimization, preclinical development, clinical testing, and/or therapeutic treatment. For example, the binding energy modeling system 100 can perform target identification to identify a biological molecule (e.g., such as a protein and/or a nucleic acid) to modulate to cause a desired therapeutic effect. The binding energy modeling system 100 can perform hit identification to discover, via computational screening and/or in vitro experimentation, compounds (e.g., such as ligands) that interact with the identified biological molecule (e.g., such as via a binding interaction). Further, the binding energy modeling system 100 can perform hit-to-lead optimization to refine initial hit compounds to improve potency, selectivity, and chemical stability of identified compounds while still maintaining desirable physiochemical properties in the compounds. Additionally, the binding energy modeling system 100 can perform lead optimization to further refine or modify the identified compounds to enhance efficacy, pharmacokinetic performance, and safety prior to preclinical testing. Further, the binding energy modeling system 100 can perform or assist in preclinical development by performing in vitro and/or in vivo assays to assess pharmacological activity, toxicity, and dosing characteristics. Additionally, clinical testing and therapeutic treatment can involve evaluation and/or application of the compound in human subjects.

The binding free energy metric 719 can be used in any of these processes. For example, during target identification, the binding energy modeling system 100 can utilize the binding free energy metric 719 to prioritize protein targets that the binding energy modeling system 100 validates through biological assays (e.g., such as enzyme activity assays, genetic knockout assays, or cell-based phenotypic assays). During hit identification, the binding energy modeling system 100 can utilize the binding free energy metric 719 to inform compound selection following experimental screens (e.g., such as high-throughput screening (HTS) fluorescence polarization assays, surface plasmon resonance (SPR), thermal shift assays (e.g., differential scanning fluorimetry), and/or fragment-based biophysical screens (e.g., such as NMR or X-ray crystallography). Further, during hit-to-lead optimization, the binding energy modeling system 100 can utilize the binding free energy metric 719 to guide structure-activity relationship (SAR) studies in conjunction with biochemical binding assays, cell viability assays, and in vitro absorption, distribution, metabolism, excretion, and toxicity (ADMET) profiling. During preclinical development, the binding energy modeling system 100 can utilize the binding free energy metric 719 alongside pharmacokinetic metrics, cytotoxicity metrics, among others to rank possible compounds. Further, in clinical testing and/or therapeutic development, the binding free energy metric 719 can be used to retrospectively correlate predicted binding affinities (e.g., that the binding energy modeling system 100 determines from the binding free energy metric 719) with pharmacodynamic data, efficacy data, to support dose selection (e.g., for in vivo administration), patient stratification, and/or mechanism-of-action validation).

In some embodiments, the binding energy modeling system 100 is part of a networking environment. For example, FIG. 8 illustrates a diagram of an example environment in which a binding energy modeling system 100 can operate in accordance with one or more embodiments.

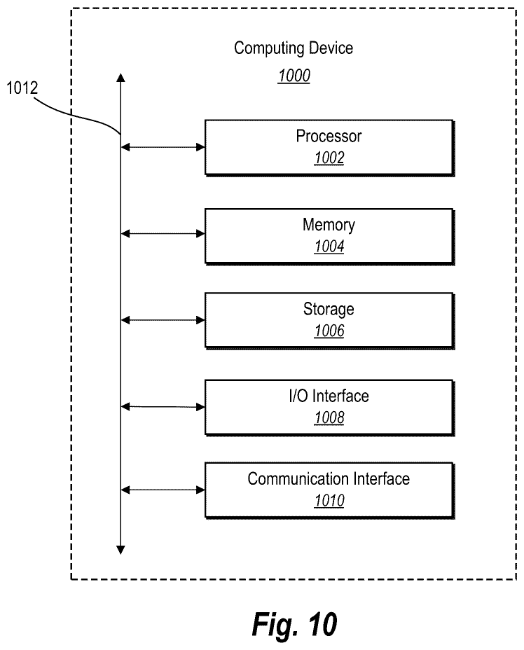

As shown in FIG. 8, the environment includes server(s) 802 (which includes a tech-bio exploration system 804 and the binding energy modeling system 100), a network 806, client device(s) 808, and testing device(s) 810. As further illustrated in FIG. 8, the various computing devices within the environment can communicate via the network 806. Although FIG. 8 illustrates the binding energy modeling system 100 being implemented by a particular component and/or device within the environment, the binding energy modeling system 100 can be implemented, in whole or in part, by other computing devices and/or components in the environment (e.g., the client device(s) 808). Additional description regarding the illustrated computing devices is provided with respect to FIG. 10 below.

As shown in FIG. 8, the server(s) 802 can include the tech-bio exploration system 804. In some embodiments, the tech-bio exploration system 804 can determine, store, generate, analyze and/or display tech-bio information including maps of biology, biology experiments from various sources, and/or machine learning tech-bio predictions. For instance, the tech-bio exploration system 804 can analyze data signals corresponding to various treatments or interventions (e.g., compounds or biologics) and the corresponding relationships in genetics, proteomics, phenomics (i.e., cellular phenotypes), and invivomics (e.g., expressions or results within a living animal). In one or more embodiments, the server(s) a binding comprises a data server. In some implementations, the server(s) comprises a communication server or a web-hosting server.

Further, the tech-bio exploration system 804 can generate and access experimental results corresponding to gene sequences, protein shapes/folding, protein/compound interactions, phenotypes resulting from various interventions or perturbations (e.g., gene knockout sequences or compound treatments), and/or in vivo experimentation on various treatments in living animals. By analyzing these signals (e.g., utilizing various machine learning models), the tech-bio exploration system 804 can generate or determine a variety of predictions and inter-relationships for improving treatments/interventions.

To illustrate, the tech-bio exploration system 804 can generate maps of biology indicating biological inter-relationships or similarities between these various input signals to discover potential new treatments. For example, the tech-bio exploration system 804 can utilize machine learning and/or maps of biology to identify a similarity between a first gene associated with disease treatment and a second gene previously unassociated with the disease based on a similarity in resulting phenotypes from gene knockout experiments. The tech-bio exploration system 804 can then identify new treatments based on the gene similarity. Similarly, the tech-bio exploration system 804 can analyze signals from a variety of sources (e.g., protein interactions, or in vivo experiments) to predict efficacious treatments based on various levels of biological data.

The tech-bio exploration system 804 can generate GUIs comprising dynamic user interface elements to convey tech-bio information and receive user input for intelligently exploring tech-bio information. Indeed, as mentioned above, the tech-bio exploration system 804 can generate GUIs displaying different maps of biology that intuitively and efficiently express complex interactions between different biological systems for identifying improved treatment solutions. Furthermore, the tech-bio exploration system 804 can also electronically communicate tech-bio information between various computing devices.

As shown in FIG. 8, the tech-bio exploration system 804 can include a system that facilitates various models or algorithms for generating maps of biology (e.g., maps or visualizations illustrating similarities or relationships between genes, proteins, diseases, compounds, and/or treatments) and discovering new treatment options over one or more networks. For example, the tech-bio exploration system 804 collects, manages, and transmits data across a variety of different entities, accounts, and devices. In some cases, the tech-bio exploration system 804 is a network system that facilitates access to (and analysis of) tech-bio information within a centralized operating system. Indeed, the tech-bio exploration system 804 can link data from different network-based research institutions to generate and analyze maps of biology.

As shown in FIG. 8, the tech-bio exploration system 804 can include a system that comprises the binding energy modeling system 100 that generates, stores, manages, transmits, and analyzes protein representations, compound representations, and molecular dynamics simulations between the protein and compound representations. For example, the binding energy modeling system 100 can generate predicted protein binding structure conformations, predicted compound binding poses, corrected protein binding structure conformations, and/or corrected compound binding structures. From the corrected protein binding structure conformations and the corrected compound binding structures, the binding energy modeling system 100 can simulate a molecular dynamics reaction and generate finding free energy metrics.

As also illustrated in FIG. 8, the environment includes the client device(s) 808. For example, the client device(s) 808 may include, but is not limited to, a mobile device (e.g., smartphone, tablet) or other type of computing device, including those explained below with reference to FIG. 10. Additionally, the client device(s) 808 can include a computing device associated with (and/or operated by) user accounts for the tech-bio exploration system 804. Moreover, the environment can include various numbers of client devices that communicate and/or interact with the tech-bio exploration system 804 and/or the binding energy modeling system 100.

Furthermore, in one or more implementations, the client device(s) 808 includes a client application. The client application can include instructions that (upon execution) cause the client device(s) 808 to perform various actions. For example, a user of a user account can interact with the client application on the client device(s) 808 to access tech-bio information, initiate a request for a binding free energy metric, for a potency metric, and/or generate GUIs comprising corrected protein binding structure conformations, corrected compound binding structures, binding free energy metrics, potency metrics, or other machine learning dataset and/or machine learning predictions/results.

As further shown in FIG. 8, the environment includes the network 806. As mentioned above, the network 806 can enable communication between components of the environment. In one or more embodiments, the network 806 may include a suitable network and may communicate using a various number of communication platforms and technologies suitable for transmitting data and/or communication signals, examples of which are described with reference to FIG. 10. Furthermore, although FIG. 8 illustrates computing devices communicating via the network 806, the various components of the environment can communicate and/or interact via other methods (e.g., communicate directly).

As mentioned previously, in one or more implementations, the binding energy modeling system 100 generates and accesses machine learning objects, such as results from biological assays, genetic and/or proteomics assays, in vivo trials, results from molecular dynamics simulation models, etc. As shown in FIG. 8, the binding energy modeling system 100 can communicate with testing device(s) 810 to obtain and then store this information. For example, the tech-bio exploration system 804 can interact with the testing device(s) 810 that include intelligent robotic devices and camera devices for generating and capturing digital images of cellular phenotypes resulting from different perturbations (e.g., genetic knockouts or compound treatments of stem cells). Similarly, the testing device(s) can include camera devices and/or other sensors (e.g., heat or motion sensors) capturing real-time information from animals as part of in vivo experimentation. The tech-bio exploration system 804 can also interact with a variety of other testing device(s) such as devices for determining, generating, or extracting gene sequences or protein information.

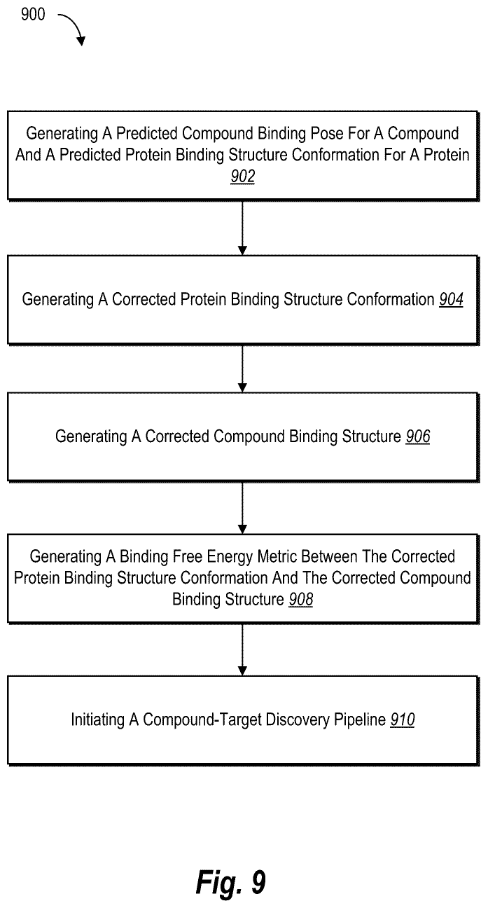

FIGS. 1-8, the corresponding text, and the examples provide a number of different systems, methods, and non-transitory computer readable media for generating a binding free energy metric for a molecular dynamics simulation between a corrected protein binding structure conformation and a corrected compound binding structure. In addition to the foregoing, embodiments can also be described in terms of flowcharts comprising acts for accomplishing a particular result. For example, FIG. 9 illustrates a flowchart of an example sequence of acts in accordance with one or more embodiments.

While FIG. 9 illustrates acts according to some embodiments, alternative embodiments may omit, add to, reorder, and/or modify any of the acts shown in FIG. 9. The acts of FIG. 9 can be performed as part of a method (e.g., a computer-implemented method). Alternatively, a non-transitory computer-readable medium can comprise instructions that, when executed by one or more processors (e.g., at least one processor), cause a computing device to perform the acts of FIG. 9. In still further embodiments, a system can perform the acts of FIG. 9. Additionally, the acts described herein may be repeated or performed in parallel with one another or in parallel with different instances of the same or similar acts.

FIG. 9 illustrates an example series of acts 900 for initiating a compound-target discovery pipeline based on generating a binding free energy metric. The series of acts 900 can include an act 902 of generating a predicted compound binding pose for a compound and a predicted protein binding structure conformation for a protein. Specifically, the act 902 can include generating, utilizing a protein-ligand structural generation machine learning model, a binding simulation between a compound and a protein, the binding simulation comprising a predicted compound binding pose for the compound and a predicted protein binding structure conformation for the protein.

Additionally, the series of acts 900 can include an act 904 of generating a corrected protein binding structure conformation. Specifically, the act 904 can include generating, utilizing a protein preparation model, a corrected protein binding structure conformation from the predicted protein binding structure conformation.

Further, the series of acts 900 can include an act 906 of generating a corrected compound binding structure. Specifically, the act 906 can include generating, utilizing a compound modification model and a re-docking simulation model, a corrected compound binding structure from the predicted compound binding pose.

In addition, the series of acts 900 can include an act 908 of generating a binding free energy metric between the corrected protein binding structure conformation and the corrected compound binding structure. Specifically, the act 908 can include generating, utilizing a molecular dynamics simulation model, a binding free energy metric between the corrected protein binding structure conformation and the corrected compound binding structure.

Moreover, the series of acts 900 can include an act 910 of initiating a compound-target discovery pipeline. Specifically, the act 910 can include initiating a compound-target discovery pipeline utilizing the binding free energy metric.

For example, the series of acts 900 can include acts to perform any of the operations described in the following clauses:
CLAUSE 1: A method, comprising:
generating, utilizing a protein-ligand structural generation machine learning model, a binding simulation between a compound and a protein, the binding simulation comprising a predicted compound binding pose for the compound and a predicted protein binding structure conformation for the protein;
generating, utilizing a protein preparation model, a corrected protein binding structure conformation from the predicted protein binding structure conformation;
generating, utilizing a compound modification model and a re-docking simulation model, a corrected compound binding structure from the predicted compound binding pose;
generating, utilizing a molecular dynamics simulation model, a binding free energy metric between the corrected protein binding structure conformation and the corrected compound binding structure; and
initiating a compound-target discovery pipeline utilizing the binding free energy metric.
CLAUSE 2: The method of clause 1, further comprising:
generating the corrected protein binding structure conformation by utilizing the protein preparation model to add hydrogen atom representations to the predicted protein binding structure conformation.
CLAUSE 3: The method of any of clauses 1-2, further comprising:
generating the corrected protein binding structure conformation by utilizing the protein preparation model to add electron representations to the predicted protein binding structure conformation.
CLAUSE 4: The method of any of clauses 1-3, further comprising:
identifying at least one of a bond order error, an aromaticity error, or a stereochemistry error for the predicted compound binding pose; and
generating an intermediate compound binding structure by generating at least one of a modified bond order, a modified aromaticity, or a modified stereochemistry from the predicted compound binding pose to account for at least one of the bond order error, the aromaticity error, or the stereochemistry error.
CLAUSE 5: The method of any of clauses 1-4, further comprising:
generating, utilizing the re-docking simulation model, the corrected compound binding structure by pairing the intermediate compound binding structure and the predicted protein binding structure conformation.
CLAUSE 6: The method of any of clauses 1-5, wherein generating the corrected compound binding structure further comprises:
generating, utilizing the compound modification model, an expected compound binding pose comprising a second stereochemistry different than a first stereochemistry corresponding to the predicted compound binding pose; and
generating a first stereochemistry error metric by comparing the predicted compound binding pose having the first stereochemistry to the predicted protein binding structure conformation.
CLAUSE 7: The method of any of clauses 1-6, further comprising:
generating a second stereochemistry error metric by comparing the expected compound binding pose having the second stereochemistry to the predicted protein binding structure conformation; and
generating the corrected compound binding structure based on the first stereochemistry error metric and the second stereochemistry error metric.
CLAUSE 8: The method of any of clauses 1-7, wherein generating the binding free energy metric further comprises:
determining a plurality of bond angles;
determining an unstable bond angle from the plurality of bond angles; and
applying a corrective force in the molecular dynamics simulation model to the unstable bond angle.
CLAUSE 9: The method of any of clauses 1-8, wherein initiating the compound-target discovery pipeline comprises:
generating, utilizing a perturbation machine learning model, a plurality of perturbation embeddings for a plurality of concentrations from functional response data measured from applying a query perturbation to a plurality of cells at the plurality of concentrations;
comparing the plurality of perturbation embeddings for the plurality of concentrations to determine a potency metric that corresponds to the query perturbation; and
comparing the potency metric to the binding free energy metric.
CLAUSE 10: The method of any of clauses 1-9, further comprising initiating the compound-target discovery pipeline by initiating a compound discovery assay based on determining the binding free energy metric.
CLAUSE 11: A method, comprising:
generating, utilizing a protein-ligand structural generation machine learning model, a binding simulation between a compound and a protein, the binding simulation comprising a predicted compound binding pose for the compound and a predicted protein binding structure conformation for the protein;
generating, utilizing a protein preparation model, a corrected protein binding structure conformation from the predicted protein binding structure conformation;
generating, utilizing a compound modification model and a re-docking simulation model, a corrected compound binding structure from the predicted compound binding pose;
generating, utilizing a molecular dynamics simulation model, a binding free energy metric between the corrected protein binding structure conformation and the corrected compound binding structure; and
providing, for display via a client device, the binding free energy metric for the protein and the compound.
CLAUSE 12: The method of clause 11, further comprising:
generating the corrected protein binding structure conformation by utilizing the protein preparation model to add hydrogen atom representations to the predicted protein binding structure conformation; and
generating the corrected protein binding structure conformation by utilizing the protein preparation model to add electron representations to the predicted protein binding structure conformation.
CLAUSE 13: The method of any of clauses 11-12, further comprising:
identifying at least one of a bond order error, an aromaticity error, or a stereochemistry error for the predicted compound binding pose; and
generating an intermediate compound binding structure by generating at least one of a modified bond order, a modified aromaticity, or a modified stereochemistry from the predicted compound binding pose to account for at least one of the bond order error, the aromaticity error, or the stereochemistry error.
CLAUSE 14: The method of any of clauses 11-13, further comprising:
generating, utilizing the re-docking simulation model, the corrected compound binding structure by pairing the intermediate compound binding structure and the predicted protein binding structure conformation.
CLAUSE 15: The method of any of clauses 11-14, wherein generating the binding free energy metric comprises:
determining a plurality of bond angles;
determining an unstable bond angle from the plurality of bond angles; and
applying a corrective force in the molecular dynamics simulation model to the unstable bond angle.
CLAUSE 16: A method, comprising:
generating, utilizing a protein-ligand structural generation machine learning model, a binding simulation between a compound and a protein, the binding simulation comprising a predicted compound binding pose for the compound and a predicted protein binding structure conformation for the protein;
generating, utilizing a protein preparation model, a corrected protein binding structure conformation from the predicted protein binding structure conformation;
generating, utilizing a compound modification model and a re-docking simulation model, a corrected compound binding structure from the predicted compound binding pose;
generating, utilizing a molecular dynamics simulation model, a binding free energy metric between the corrected protein binding structure conformation and the corrected compound binding structure.
CLAUSE 17: The method of clause 16, further comprising:
generating the corrected protein binding structure conformation by utilizing the protein preparation model to add hydrogen atom representations to the predicted protein binding structure conformation; and
generating the corrected protein binding structure conformation by utilizing the protein preparation model to add electron representations to the predicted protein binding structure conformation.
CLAUSE 18: The system of any of clauses 16-17, further comprising:
identifying at least one of a bond order error, an aromaticity error, or a stereochemistry error for the predicted compound binding pose; and
generating an intermediate compound binding structure by generating at least one of a modified bond order, a modified aromaticity, or a modified stereochemistry from the predicted compound binding pose to account for at least one of the bond order error, the aromaticity error, or the stereochemistry error.
CLAUSE 19: The method of any of clauses 16-18, further comprising:
generating, utilizing the re-docking simulation model, the corrected compound binding structure by pairing the intermediate compound binding structure and the predicted protein binding structure conformation.
CLAUSE 20: The method of any of clauses 16-19, further comprising generating the binding free energy metric by:
determining a plurality of bond angles;
determining an unstable bond angle from the plurality of bond angles; and
applying a corrective force in the molecular dynamics simulation model to the unstable bond angle.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 10 illustrates a block diagram of an example computing device 1000 that may be configured to perform one or more of the processes described above. One will appreciate that one or more computing devices, such as the computing device 1000 may represent the computing devices described above. In one or more embodiments, the computing device 1000 may be a mobile device (e.g., a mobile telephone, a smartphone, a PDA, a tablet, a laptop, a camera, a tracker, a watch, a wearable device, etc.). In some embodiments, the computing device 1000 may be a non-mobile device (e.g., a desktop computer or another type of client device). Further, the computing device 1000 may be a server device that includes cloud-based processing and storage capabilities.

As shown in FIG. 10, the computing device 1000 can include one or more processor(s) 1002, memory 1004, a storage device 1006, input/output interfaces 1008 (or “I/O interfaces 1008”), and a communication interface 1010, which may be communicatively coupled by way of a communication infrastructure (e.g., bus 1012). While the computing device 1000 is shown in FIG. 10, the components illustrated in FIG. 10 are not intended to be limiting. Additional or alternative components may be used in other embodiments. Furthermore, in certain embodiments, the computing device 1000 includes fewer components than those shown in FIG. 10. Components of the computing device 1000 shown in FIG. 10 will now be described in additional detail.

In particular embodiments, the processor(s) 1002 includes hardware for executing instructions, such as those making up a computer program. As an example, and not by way of limitation, to execute instructions, the processor(s) 1002 may retrieve (or fetch) the instructions from an internal register, an internal cache, memory 1004, or a storage device 1006 and decode and execute them.

The computing device 1000 includes memory 1004, which is coupled to the processor(s) 1002. The memory 1004 may be used for storing data, metadata, and programs for execution by the processor(s). The memory 1004 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1004 may be internal or distributed memory.

The computing device 1000 includes a storage device 1006 includes storage for storing data or instructions. As an example, and not by way of limitation, the storage device 1006 can include a non-transitory storage medium described above. The storage device 1006 may include a hard disk drive (HDD), flash memory, a Universal Serial Bus (USB) drive or a combination these or other storage devices.

As shown, the computing device 1000 includes one or more I/O interfaces 1008, which are provided to allow a user to provide input to (such as user strokes), receive output from, and otherwise transfer data to and from the computing device 1000. These I/O interfaces 1008 may include a mouse, keypad or a keyboard, a touch screen, camera, optical scanner, network interface, modem, other known I/O devices or a combination of such I/O interfaces 1008. The touch screen may be activated with a stylus or a finger.

The I/O interfaces 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1008 are configured to provide graphical data to a display for presentation to a user. The graphical data may be representative of one or more graphical user interfaces and/or any other graphical content as may serve a particular implementation.

The computing device 1000 can further include a communication interface 1010. The communication interface 1010 can include hardware, software, or both. The communication interface 1010 provides one or more interfaces for communication (such as, for example, packet-based communication) between the computing device and one or more other computing devices or one or more networks. As an example, and not by way of limitation, communication interface 1010 may include a network interface controller (NIC) or network adapter for communicating with an Ethernet or other wire-based network or a wireless NIC (WNIC) or wireless adapter for communicating with a wireless network, such as a WI-FI. The computing device 1000 can further include a bus 1012. The bus 1012 can include hardware, software, or both that connects components of computing device 1000 to each other.

In one or more implementations, various computing devices can communicate over a computer network. This disclosure contemplates any suitable network. As an example, and not by way of limitation, one or more portions of a network may include an ad hoc network, an intranet, an extranet, a virtual private network (“VPN”), a local area network (“LAN”), a wireless LAN (“WLAN”), a wide area network (“WAN”), a wireless WAN (“WWAN”), a metropolitan area network (“MAN”), a portion of the Internet, a portion of the Public Switched Telephone Network (“PSTN”), a cellular telephone network, or a combination of two or more of these.

In particular embodiments, the computing device 1000 can include a client device that includes a requester application or a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user at the client device may enter a Uniform Resource Locator (“URL”) or other address directing the web browser to a particular server (such as server), and the web browser may generate a Hyper Text Transfer Protocol (“HTTP”) request and communicate the HTTP request to server. The server may accept the HTTP request and communicate to the client device one or more Hyper Text Markup Language (“HTML”) files responsive to the HTTP request. The client device may render a webpage based on the HTML files from the server for presentation to the user. This disclosure contemplates any suitable webpage files. As an example, and not by way of limitation, webpages may render from HTML files, Extensible Hyper Text Markup Language (“XHTML”) files, or Extensible Markup Language (“XML”) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a webpage encompasses one or more corresponding webpage files (which a browser may use to render the webpage) and vice versa, where appropriate.

In particular embodiments, the tech-bio exploration system 804 may include a variety of servers, sub-systems, programs, modules, logs, and data stores. In particular embodiments, the tech-bio exploration system 804 may include one or more of the following: a web server, action logger, API-request server, transaction engine, cross-institution network interface manager, notification controller, action log, third-party-content-object-exposure log, inference module, authorization/privacy server, search module, user-interface module, user-profile (e.g., provider profile or requester profile) store, connection store, third-party content store, or location store. The tech-bio exploration system 804 may also include suitable components such as network interfaces, security mechanisms, load balancers, failover servers, management-and-network-operations consoles, other suitable components, or any suitable combination thereof. In particular embodiments, the tech-bio exploration system 804 may include one or more user-profile stores for storing user profiles and/or account information for credit accounts, secured accounts, secondary accounts, and other affiliated financial networking system accounts. A user profile may include, for example, biographic information, demographic information, financial information, behavioral information, social information, or other types of descriptive information, such as interests, affinities, or location.

The web server may include a mail server or other messaging functionality for receiving and routing messages between the tech-bio exploration system 804 and one or more client devices. An action logger may be used to receive communications from a web server about a user's actions on or off the tech-bio exploration system 804. In conjunction with the action log, a third-party-content-object log may be maintained of user exposures to third-party-content objects. A notification controller may provide information regarding content objects to a client device. Information may be pushed to a client device as notifications, or information may be pulled from a client device responsive to a request received from the client device. Authorization servers may be used to enforce one or more privacy settings of the users of the tech-bio exploration system 804. A privacy setting of a user determines how particular information associated with a user can be shared. The authorization server may allow users to opt in to or opt out of having their actions logged by the tech-bio exploration system 804 or shared with other systems, such as, for example, by setting appropriate privacy settings. Third-party-content-object stores may be used to store content objects received from third parties. Location stores may be used for storing location information received from a client device associated with users.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

1. A method, comprising:
generating, utilizing a protein-ligand structural generation machine learning model, a binding simulation between a compound and a protein, the binding simulation comprising a predicted compound binding pose for the compound conformed to bind to a predicted protein binding structure conformation for the protein;
generating, utilizing a protein preparation model, a corrected protein binding structure conformation from the predicted protein binding structure conformation of the binding simulation;
generating, utilizing a compound modification model and a re-docking simulation model, a corrected compound binding structure from the predicted compound binding pose of the binding simulation;
generating, utilizing a molecular dynamics simulation model, a binding free energy metric between the corrected protein binding structure conformation generated from the predicted protein binding structure conformation of the binding simulation and the corrected compound binding structure generated from the predicted compound binding pose of the binding simulation; and
initiating a compound-target discovery pipeline utilizing the binding free energy metric.

2. The method of claim 1, further comprising:
generating the corrected protein binding structure conformation by utilizing the protein preparation model to add hydrogen atom representations to the predicted protein binding structure conformation.

3. The method of claim 1, further comprising generating the corrected protein binding structure conformation by utilizing the protein preparation model to add electron representations to the predicted protein binding structure conformation.

4. The method of claim 1, further comprising:
identifying at least one of a bond order error, an aromaticity error, or a stereochemistry error for the predicted compound binding pose; and
generating an intermediate compound binding structure by generating at least one of a modified bond order, a modified aromaticity, or a modified stereochemistry from the predicted compound binding pose to account for at least one of the bond order error, the aromaticity error, or the stereochemistry error.

5. The method of claim 4, further comprising:
generating, utilizing the re-docking simulation model, the corrected compound binding structure by pairing the intermediate compound binding structure and the predicted protein binding structure conformation.

6. The method of claim 1, wherein generating the corrected compound binding structure comprises:
identifying an expected compound binding pose comprising a second stereochemistry different than a first stereochemistry corresponding to the predicted compound binding pose; and
generating a first stereochemistry error metric by comparing the predicted compound binding pose having the first stereochemistry to the predicted protein binding structure conformation.

7. The method of claim 6, further comprising:
generating a second stereochemistry error metric by comparing the expected compound binding pose having the second stereochemistry to the predicted protein binding structure conformation; and
generating the corrected compound binding structure based on the first stereochemistry error metric and the second stereochemistry error metric.

8. The method of claim 1, wherein generating the binding free energy metric comprises:
determining a plurality of bond angles;
determining an unstable bond angle from the plurality of bond angles; and
applying a corrective force in the molecular dynamics simulation model to the unstable bond angle.

9. The method of claim 1, wherein initiating the compound-target discovery pipeline comprises:
generating, utilizing a perturbation machine learning model, a plurality of perturbation embeddings for a plurality of concentrations from functional response data measured from applying a query perturbation to a plurality of cells at the plurality of concentrations;
comparing the plurality of perturbation embeddings for the plurality of concentrations to determine a potency metric that corresponds to the query perturbation; and
comparing the potency metric to the binding free energy metric.

10. The method of claim 1, further comprising initiating the compound-target discovery pipeline by initiating a compound discovery assay based on determining the binding free energy metric.

11. A system comprising:
at least one processor; and
at least one non-transitory computer-readable storage medium storing instructions that, when executed by the at least one processor, cause the system to:
generate, utilizing a protein-ligand structural generation machine learning model, a binding simulation between a compound and a protein, the binding simulation comprising a predicted compound binding pose for the compound conformed to bind to and a predicted protein binding structure conformation for the protein;
generate, utilizing a protein preparation model, a corrected protein binding structure conformation from the predicted protein binding structure conformation of the binding simulation;
generate, utilizing a compound modification model and a re-docking simulation model, a corrected compound binding structure from the predicted compound binding pose of the binding simulation;
generate, utilizing a molecular dynamics simulation model, a binding free energy metric between the corrected protein binding structure conformation generated from the predicted protein binding structure conformation of the binding simulation and the corrected compound binding structure generated from the predicted compound binding pose of the binding simulation; and
provide, for display via a client device, the binding free energy metric for the protein and the compound.

12. The system of claim 11, further comprising instructions that, when executed by the at least one processor, cause the system to:
generate the corrected protein binding structure conformation by utilizing the protein preparation model to add hydrogen atom representations to the predicted protein binding structure conformation; and
generate the corrected protein binding structure conformation by utilizing the protein preparation model to add electron representations to the predicted protein binding structure conformation.

13. The system of claim 11, further comprising instructions that, when executed by the at least one processor, cause the system to:
identify at least one of a bond order error, an aromaticity error, or a stereochemistry error for the predicted compound binding pose; and
generate an intermediate compound binding structure by generating at least one of a modified bond order, a modified aromaticity, or a modified stereochemistry from the predicted compound binding pose to account for at least one of the bond order error, the aromaticity error, or the stereochemistry error.

14. The system of claim 13, further comprising instructions that, when executed by the at least one processor, cause the system to generate, utilizing the re-docking simulation model, the corrected compound binding structure by pairing the intermediate compound binding structure and the predicted protein binding structure conformation.

15. The system of claim 11, further comprising instructions that, when executed by the at least one processor, cause the system to generate the binding free energy metric by:
determining a plurality of bond angles;
determining an unstable bond angle from the plurality of bond angles; and
applying a corrective force in the molecular dynamics simulation model to the unstable bond angle.

16. A non-transitory computer-readable medium storing instructions that, when executed by at least one processor, cause a computing device to:
generate, utilizing a protein-ligand structural generation machine learning model, a binding simulation between a compound and a protein, the binding simulation comprising a predicted compound binding pose for the compound conformed to bind to a predicted protein binding structure conformation for the protein;
generate, utilizing a protein preparation model, a corrected protein binding structure conformation from the predicted protein binding structure conformation of the binding simulation;
generate, utilizing a compound modification model and a re-docking simulation model, a corrected compound binding structure from the predicted compound binding pose of the binding simulation; and
generate, utilizing a molecular dynamics simulation model, a binding free energy metric between the corrected protein binding structure conformation generated from the predicted protein binding structure conformation of the binding simulation and the corrected compound binding structure generated from the predicted compound binding pose of the binding simulation.

17. The non-transitory computer-readable medium of claim 16, further comprising instructions that, when executed by the at least one processor, cause the computing device to:
generate the corrected protein binding structure conformation by utilizing the protein preparation model to add hydrogen atom representations to the predicted protein binding structure conformation; and
generate the corrected protein binding structure conformation by utilizing the protein preparation model to add electron representations to the predicted protein binding structure conformation.

18. The non-transitory computer-readable medium of claim 16, further comprising instructions that, when executed by the at least one processor, cause the computing device to:
identify at least one of a bond order error, an aromaticity error, or a stereochemistry error for the predicted compound binding pose; and
generating an intermediate compound binding structure by generating at least one of a modified bond order, a modified aromaticity, or a modified stereochemistry from the predicted compound binding pose to account for at least one of the bond order error, the aromaticity error, or the stereochemistry error.

19. The non-transitory computer-readable medium of claim 18, further comprising instructions that, when executed by the at least one processor, cause the computing device to generate, utilizing the re-docking simulation model, the corrected compound binding structure by pairing the intermediate compound binding structure and the predicted protein binding structure conformation.

20. The non-transitory computer-readable medium of claim 16, further comprising instructions that, when executed by the at least one processor, cause the computing device to generate the binding free energy metric by:
determining a plurality of bond angles;
determining an unstable bond angle from the plurality of bond angles; and
applying a corrective force in the molecular dynamics simulation model to the unstable bond angle.
